(19) United States
(12) Patent Application Publication
(10) Pub. No.: US 2025/0161182 A1
(43) Pub. Date: May 22, 2025

(54) ANTIMICROBIAL COMPOSITIONS

(71) Applicant: BIO-ACTIVES SYNERGIO LTD, Jerusalem (IL)

(72) Inventors: Tova SILBERSTEIN, Jerusalem (IL); Alexander BESONOV, Rehovot (IL); Rachel LUTZ, Gush etzion (IL); Sabrina BARCHICHAT, Jerusalem (IL); Tal SHALEV, Tzur Hadassah (IL); Tamar DVASH, Kibbutz Tzuba (IL)

(21) Appl. No.: 18/833,244

(22) PCT Filed: Jan. 27, 2022
(86) PCT No.: PCT/IL2022/050123

§ 371 Date: Jul. 25, 2024

(51) Int. Cl. A61K 8/49; A61Q 5/00; A61Q 11/00; A61Q 19/00

(52) U.S. Cl. CPC A61K 8/498; A61Q 5/00; A61Q 11/00; A61Q 19/00; A61K 2800/524; A61K 2800/592; A61K 2800/70; A61K 2800/74

(57) ABSTRACT

The disclosure generally concerns antimicrobial compositions, specifically antimicrobial synergistic combinations of components obtained from natural sources, more specifically compositions of at least one 4-pyrone derivative and one or more plant extracts.

### TECHNOLOGICAL FIELD

This disclosure generally concerns antimicrobial compositions, more specifically antimicrobial synergistic combinations of components obtained from natural sources.

### REFERENCES

References considered to be relevant as background to the presently disclosed subject matter are listed below:
[1] WO 2012/077119
[2] WO 2017/191629

Acknowledgement of the above references herein is not to be inferred as meaning that these are in any way relevant to the patentability of the presently disclosed subject matter.

### BACKGROUND

Various cosmetic, personal care and pharmaceutical compositions comprise preservatives in order to prevent antimicrobial growth or proliferation, as well as increase the shelf-life of products. Public concern about the safety of synthetic preservatives used in such products, especially regarding their accumulation and subsequent health effect, have driven health authorities to reduce the applied concentrations or even ban synthetic preservatives. Alternatives such as plant-based antimicrobial substances are in the focus of many researches, however due to low potency, narrow range and high prices, they are rarely used to replace synthetic preservatives.

### GENERAL DESCRIPTION

Various naturally-based components are known to have some antimicrobial activity; however, such may often prove to be insufficient against various microbiological contaminants. The inventors of the invention disclosed herein have surprisingly found that compositions of 4-pyrone derivatives (such as maltol) and extract of plants from specific genera exhibit biological activity, i.e. antimicrobial activity, which is superior to the activity demonstrated for each component individually and which is at least comparable, and at times even superior, to chemical (i.e. non-natural) alternatives known for the same use.

Thus, in one of its aspects, this disclosure provides an antimicrobial composition comprising at least one 4-pyrone derivative, and an extract from at least one plant species.

The 4-pyrone derivative may have a structure of formula (I):

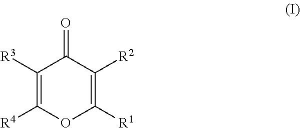

wherein:
R¹ is selected from H, C₁₋₆alkyl, C₁₋₆hydroxyalkyl, C₁₋₆acyl, and C₂₋₆alkenyl;
R² is selected from H, OH, —O—C(O)—R⁵;
R³ is H or OH;
R⁴ is H or OH;
R⁵ is selected from H, C₁₋₆alkyl, C₁₋₆hydroxyalkyl, and a phenyl (optionally substituted by one, two or three substituents independently selected from H, OH, halo, C₁₋₆alkyl, C₂₋₆alkenyl, and —O—C(O)—(C₁₋₆alkyl));
or a salt or a hydrate thereof,
provided that at any instance at least one of R¹, R², R³ and R⁴ is not H.

In some embodiments,
R¹ is selected from H, C₁₋₆alkyl, C₁₋₆hydroxyalkyl, and C₁₋₆acyl;
R² is H or OH;
R³ is H or OH;
R⁴ is H or OH;
provided that at any instance at least one of R¹, R², R³ and R⁴ is not H.

According to some embodiments, the 4-pyrone derivative is selected from:
3-hydroxy-2-methyl-4H-pyran-4-one (maltol),
5-hydroxy-2-methyl-4H-pyran-4-one (allomaltol),
5-hydroxy-2-(hydroxymethyl)-4H-pyran-4-one (kojic acid),
2-ethyl-3-hydroxy-4H-pyran-4-one,
3-hydroxy-2-propylpyran-4-one,
3-hydroxy-2-propan-2-ylpyran-4-one,
2-acetyl-3-hydroxypyran-4-one,
2-ethyl-5-hydroxypyran-4-one,
3-hydroxy-2-(2-propen-1-yl)-4H-Pyran-4-one,
3-hydroxypyran-4-one,
2-hex-2-enyl-3-hydroxypyran-4-one,
3,5-dihydroxy-2-methyl-4H-pyran-4-one (5-Hydroxymaltol),
2-methyl-4-oxo-4H-pyran-3-yl isobutyrate (Maltol isobutyrate),
2-methyl-3-(1-oxopropoxy)-4H-pyran-4-one (Maltol propionate),
2-methyl-4-oxo-4H-pyran-3-yl butyrate (Maltol butyrate),
(2-methyl-4-oxopyran-3-yl) acetate,
(2-ethyl-4-oxopyran-3-yl) 2-methylpropanoate,
2-methyl-4-oxo-4H-pyran-3-yl 2-hydroxybenzoate (Salicylic acid maltol ester),
2-methyl-4-oxo-4H-pyran-3-yl lactate (Maltol lactate),
2-methyl-4-oxo-4H-pyran-3-yl 2-(acetyloxy)benzoate (Aspalatone),
(Z)-(2-methyl-4-oxopyran-3-yl)-2-methylpent-2-enoate, and
(E)-(2-methyl-4-oxopyran-3-yl)-2-methylpent-2-enoate;
and any combination or mixture thereof.

According to further embodiments, the 4-pyrone derivative is selected from the following compounds:

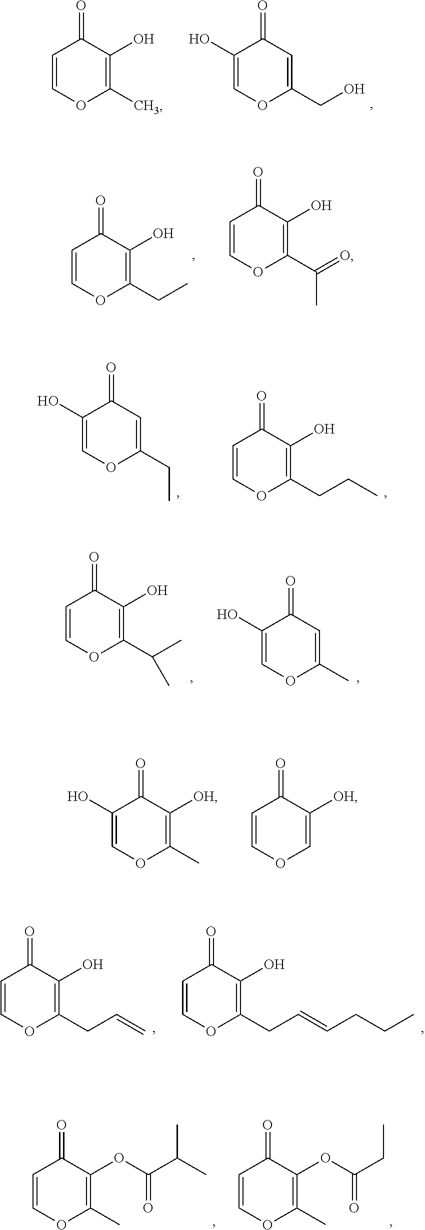

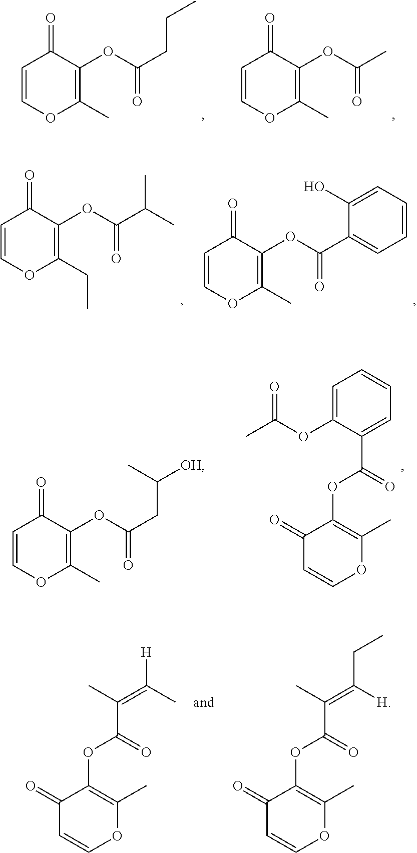

According to some embodiments, the 4-pyrone derivative is maltol.

According to other embodiments, the 4-pyrone derivative is 5-hydroxy-2-(hydroxymethyl)-4H-pyran-4-one (kojic acid).

According to some other embodiments, the 4-pyrone derivative is 2-ethyl-3-hydroxy-4H-pyran-4-one.

By some embodiments, the 4-pyrone derivative is obtained from a natural source or a non-natural source (i.e. synthetic 4-pyrone derivative). By some other embodiments, the 4-pyrone derivative is obtained from a natural source, e.g. a plant source or a non-plant natural source. The plant source from which the 4-pyrone derivative, for example maltol, can be obtained may, by some embodiments, be selected from plants of the genus Larix, plants of the genus Pinus, malted or fermented grains (such as barley, wheat, rice, corn, quinoa, etc.), fermented sugarcane, roasted legumes (e.g. beans, peanuts, fava beans, peas, chickpeas, lentils, lupins, soybean, tamarind, carob, etc.), roasted cocoa beans, roasted coffee beans, etc. and any mixture thereof. It should be appreciated that the 4-pyrone derivative(s) may be obtained from two or more different plant sources or can be a mixture or a combination of the 4-pyrone derivative(s) obtained from different plant sources. Typically, the 4-pyrone derivative is obtained from the plant source by extraction, for example, an extract of fermented sugarcane.

According to preferred embodiments, the 4-pyrone derivative is obtained from a plant source different from the at least one plant species. By some embodiments, the 4-pyron derivative is obtained as an extract of fermented sugarcane.

According to other embodiments, the 4-pyrone derivative is obtained from a non-natural source, i.e. synthetically obtained.

In other embodiments, each or both of the 4-pyrone derivative and the at least one plant extract is obtained commercially.

Compositions of this disclosure may comprise, by some embodiments, at least 0.001 wt % 4-pyrone derivative. In some embodiments, the composition comprises between about 0.001 and 5 wt % of 4-pyrone derivative. In some embodiments, the compositions comprise between about 0.001 and 4 wt %, between about 0.001 and 3 wt %, between about 0.001 and 2 wt %, or even between about 0.001 and 1 wt % of 4-pyrone derivative. In other embodiments, the compositions comprise between about 0.005 and 5 wt %, between about 0.01 and 5 wt %, between about 0.03 and 5 wt %, or even between about 0.05 and 5 wt % of 4-pyrone derivative. In some other embodiments, the compositions comprise between about 0.005 and 4 wt %, between about 0.01 and 3 wt %, between about 0.03 and 2 wt %, or even between about 0.05 and 1 wt % of 4-pyrone derivative.

The compositions of the present disclosure comprise a combination of at least one 4-pyrone derivative and one or more plant extract.

As used herein, the term extract refers to an active ingredient or fraction isolated from a plant, typically by solvent extraction, although other extraction techniques known per-se are also contemplated to be within the scope of this disclosure. The extraction procedure for obtaining any of the plant extracts employed in accordance with the present disclosure, unless otherwise indicated, may be carried out in any commonly used technique and variation known in the art.

Plant extracts used for preparing the compositions of this disclosure may be prepared prior to formulation, in advance of formulation or may be commercially available. The extracts may be used without further purification or can undergo various purifications steps in order to increase their purity.

Compositions of this disclosure may comprise, by some embodiments, at least 0.0001 wt % plant extract from said at least one plant species. In some embodiments, the compositions comprise between about 0.0001 and 5 wt % of said plant extract. In some embodiments, the compositions comprise between about 0.0001 and 4 wt %, between about 0.0001 and 3 wt %, or even between about 0.0001 and 2 wt % of the plant extract. In other embodiments, the compositions comprise between about 0.005 and 5 wt %, between about 0.001 and 5 wt %, between about 0.005 and 5 wt %, between about 0.01 and 5 wt %, or even between about 0.05 wt % and 5 wt % of said plant extract. In some other embodiments, the compositions comprise between about 0.0001 and 4 wt %, between about 0.0005 and 3 wt %, between about 0.01 and 2.5 wt %, or even between about 0.05 and 2 wt % of said plant extract.

In the compositions of this disclosure, the weight-to-weight ratio (wt/wt) between the at least one 4-pyrone derivative and the at least one plant extract may range between 1:100 and 600:1 (4-pyrone derivative:plant extract). By some other embodiments, the weight-to-weight ratio between the 4-pyrone derivative and the at least one plant extract range between 1:100 and 400:1, between 1:100 and 300:1, between 1:100 and 200:1, or even between 1:100 and 100:1.

In some embodiments, the weight-to-weight ratio between the 4-pyrone derivative and the at least one plant extract is about 1:100, 1:75, 1:50, 1:25, 1:20, 1:15, 1:10, 1:8, 1:7, 1:6, 1:5, 1:4, 1:3, 1:2, or about 1:1. In other embodiments, weight-to-weight ratio between the 4-pyrone derivative and the at least one plant extract is about 400:1, 350:1, 300:1, 250:1, 200:1, 150:1, 100:1, 75:1, 50:1, 40:1, 30:1, 25:1, 20:1, 10:1, 9:1, 8:1, 7:1, 6:1, 5:1, 4:1, 3:1, 2:1, or even 1:1.

In the compositions of this disclosure, the weight-to-weight ratio (wt/wt) between the at least one 4-pyrone derivative and the at least one plant extract may range between 1:5 and 600:1 (4-pyrone derivative:plant extract).

In some embodiments, the weight ratio between the 4-pyrone derivative and the at least one plant extract may be between 1:5 and 400:1.

In other embodiments, the weight ratio between the 4-pyrone derivative and the at least one plant extract may be between 1:5 and 300:1.

In other embodiments, the weight ratio between the 4-pyrone derivative and the at least one plant extract may be between 1:5 and 200:1.

In some other embodiments, the weight ratio between the 4-pyrone derivative and the at least one plant extract may be between 1:5 and 100:1.

According to some embodiments, the at least one plant species is selected from plants of the following genera: Styrax (a Styrax extract), Myroxylon (a Miroxylon extract), Myrocarpus (a Myrocarpus extract), Nigella (a Nigella extract), Cuminum (a Cuminum extract), Zingiber (a Zingiber extract), Cinnamomum (a Cinnamomum extract), Paeonia (a Paeonia extract), Terminalia (a Terminalia extract), Commiphora (a Commiphora extract), Boswellia (a Boswellia extract), Dipterocarpus (a Dipterocarpus extract), Copaiba (a Copaiba extract), Thymus (a Thymus extract), Origanum (an Origanum extract), Cymbopogon (a Cymbopogon extract), Anethum (a Anethum extract), Syzygium (a Syzigium extract), Magnolia (a Magnolia extract), Malpighia (a Malpighia extract), Pimenta (a Pimenta extract), Salvia (a Salvia extract), and any mixture or combination thereof.

It should be further noted that the present disclosure also contemplates compositions comprising mixtures or combinations of such extracts, whether prepared and formulated individually or prepared in one-pot from a mixture of plant sources (plant parts).

The genus Styrax contains a group of small trees or shrubs in the family Styracaceae, which often secrete a gum-like resin containing at least benzoic acid, coniferyl benzoate and other compounds. The gum-like resin or tree bark is often called “benzoin resin”. It should be appreciated that the extract may be an extract of more than one plant selected within the genus.

In some embodiments, the at least one plant extract is a Styrax extract, obtained from at least one plant of the genus Styrax may be selected from Styrax agrestis, Styrax americanus, Styrax argenteus, Styrax argentifolius, Styrax argyrophyllus, Styrax bashanensis, Styrax benzoides, Styrax benzoin, Styrax calvescens, Styrax camporum, Styrax chinensis, Styrax chrysocarpus, Styrax confuses, Styrax crotonoides, Styrax dasyanthus, Styrax faberi, Styrax ferax, Styrax ferrugineus, Styrax formosanus, Styrax foveolaria, Styrax fraserensis, Styrax grandiflorus, Styrax grandifolius, Styrax hainanensis, Styrax hemsleyanus, Styrax hookeri, Styrax huanus, Styrax jaliscana, Styrax japonicas, Styrax limpritchii, Styrax litseoides, Styrax loxensis, Styrax macranthus, Styrax macrocarpus, Styrax martii, Styrax mathewsii, Styrax obassia, Styrax odoratissimus, Styrax officinalis, Styrax paralleloneurus (Sumatra benzoin), Styrax parvifolium, Styrax perkinsiae, Styrax peruvianum, Styrax philadelphoides, Styrax platanifolius, Styrax pohlii, Styrax portoricensis, Styrax redivivus, Styrax roseus, Styrax rugosus, Styrax schweliense, Styrax serrulatus, Styrax shiraianum, Styrax socialis, Styrax suberifolius, Styrax supaii, Styrax tafelbergensis, Styrax tolu, Styrax tonkinensis (Siam Benzoin), Styrax veitchiorum, Styrax vilcabambae, Styrax wilsonii, Styrax wuyuanensis, Styrax zhejiangensis and mixtures thereof.

In some embodiments, the plant of the genus Styrax is selected from Styrax paralleloneurus (Sumatra benzoin), Styrax tonkinensis (Siam Benzoin), Styrax formosanus, Styrax peruvianum, Styrax tolu and mixtures thereof.

In some other embodiments, the plant of the genus Styrax is Styrax paralleloneurus (Sumatra benzoin).

In other embodiments, the plant of the genus Styrax is Styrax tonkinensis (Siam Benzoin).

In further embodiments, the plant of the genus Styrax is Styrax formosanus.

In some further embodiments, the plant of the genus Styrax is Styrax peruvianum.

In yet further embodiments, the plant of the genus Styrax is Styrax tolu.

In other embodiments, the Styrax extract is obtained from the resin and/or bark of Styrax paralleloneurus and/or Styrax tonkinensis.

According to some embodiments, the Styrax extract is obtained by mixing the resin and/or bark with a suitable solvent, typically a hydrophobic solvent, such as at least one oil (e.g. paraffinic oil, triglycerides, non-triglyceride oils, etc.), pentane, hexane, cyclohexane, heptane, octane, dichloromethane, dichloroethane, chloroform, etc.

The genus Myroxylon contains a group of small trees in the family Fabaceae, which often secrete a gum-like resin containing at least benzoic acid, coniferyl benzoate and other compounds.

In some embodiments, the at least one plant extract is a Myroxylon extract, obtained from the plant of the genus Myroxylon selected from Myroxylon balsamum and Myroxylon peruiferum. The Myroxylon extract can be obtained from the resin or bark of the plant.

The genus Myrocarpus contains a group of small trees also in the family Fabaceae, and its gum-like resin contains at least benzoic acid, coniferyl benzoate and other compounds.

In some embodiments, the at least one plant extract is a Myrocarpus extract, obtained from the plant of the genus Myrocarpus selected from Myrocarpus fastigiatus, Myrocarpus frondosus, Myrocarpus leprosus and Myrocarpus venezuelensis. According to some embodiments, the Myrocarus extract is a Myrocarpus fastigiatus extract. The Myrocarpus extract can be obtained from the resin or bark of the plant.

In some embodiments, the composition comprises at least one of Styrax extract, Myroxylon extract and/or Myrocarpus extract. In such embodiments, the plant extract may comprise total amount of cinnamic acid, cinnamic acid derivatives and benzoic acid derivatives in the at least one plant extract is at least 0.001, 0.01, or even at least 0.1 wt %. In other embodiments, the total amount of cinnamic acid, cinnamic acid derivatives and benzoic acid derivatives in the at least one plant extract may be at most 80 wt %. In some other embodiments, the total amount of cinnamic acid, cinnamic acid derivatives and benzoic acid derivatives in the at least one plant extract may be at most 75 wt %, 70 wt %, 65 wt %, 60 wt %, 55 wt %, 50 wt %, 45 wt %, 40 wt %, 35 wt %, 30 wt %, 25 wt %, or even 20 wt %. In some other embodiments, the total amount of cinnamic acid, cinnamic acid derivatives and benzoic acid derivatives in the at least one plant extract may be between about 0.001 and 80 wt %, between about 0.01 and 80 wt %, between about 0.1 and 80 wt %, or even between about 1 and 80 wt %. In yet other embodiments, the total amount of cinnamic acid, cinnamic acid derivatives and benzoic acid derivatives in the at least one plant extract may be between about 0.001 and 75 wt %, between about 0.001 and 70 wt %, between about 0.001 and 65 wt %, between about 0.001 and 60 wt %, between about 0.001 and 55 wt %, between about 0.001 and 50 wt %, between about 0.001 and 45 wt %, between about 0.001 and 40 wt %, between about 0.001 and 35 wt %, between about 0.001 and 30 wt %, between about 0.001 and 25 wt %, or even between about 0.001 and 20 wt %.

The term derivative refers to a chemically modified compound derived from a parent compound (e.g. cinnamic acid or benzoic acid) that differs from the parent compound by one or more elements, substituents and/or functional groups such that the derivative has the same or similar properties/activities as the parent compound, as defined herein.

In some embodiments, the cinnamic acid derivative is selected from P-coumaryl cinnamate, coniferyl cinnamate, cinnamyl cinnamate, benzyl cinnamate, cinnamic acid esters, etc., and combinations thereof.

In other embodiments, the benzoic acid derivative is selected from coniferyl benzoate, cinnamyl benzoate, P-coumaryl benzoate, benzoic acid esters, etc., and combinations thereof.

The at least one plant extract may further comprise various terpenes and terpenoids, as well as other phenolic derivatives, such as pinoresinol.

The genus Nigella is a genus of annual plants in the family Ranunculaceae, which includes, inter alia, the species Nigella arvensis, Nigella carpatha, Nigella damascena, Nigella degenii, Nigella deserti, Nigella doerfleri, Nigella elata, Nigella fumariifola, Nigella hispanica, Nigella latisecta, Nigella nigellastrum, Nigella orientalis, Nigella oxypetala, Nigella papillosa, Nigella sativa, Nigella segetalis, Nigella stricta and Nigella unguicularis.

In some embodiments, the Nigella extract is an extract of Nigella sativa. In other embodiments, the Nigella extract is an extract of Nigella sativa seeds.

The genus Cuminum is a genus flowering plants in the family Apiaceae, which includes, inter alia, the species Cuminum borszczowii, Cuminum cyminum, Cuminum setifolium, and Cuminum sudanense.

In some embodiments, the Cuminum extract is an extract of Cuminum cyminum. In other embodiments, the Cuminum extract is an extract of Cuminum cyminum seeds.

The genus Zingiber includes, inter alia, the species Zingiber acuminatum, Zingiber albiflorum, Zingiber apoense, Zingiber argenteum, Zingiber atrorubens, Zingiber aurantiacum, Zingiber banhaoense, Zingiber barbatum, Zingiber bisectum, Zingiber brachystachys, Zingiber bradleyanum, Zingiber brevifolium, Zingiber bulusanense, Zingiber callianthus, Zingiber capitatum, Zingiber cernuum, Zingiber chantaranothaii, Zingiber chlorobracteatum, Zingiber chrysanthum, Zingiber chrysostachys, Zingiber citriodorum, Zingiber clarkei, Zingiber cochleariforme, Zingiber collinsii, Zingiber coloratum, Zingiber cornubracteatum, Zingiber corallinum, Zingiber curtisii, Zingiber cylindricum, Zingiber densissimum, Zingiber eberhardtii, Zingiber eborinum, Zingiber elatior, Zingiber elatum, Zingiber ellipticum, Zingiber flagelliforme, Zingiber flammeum, Zingiber flavomaculosum, Zingiber flavovirens, Zingiber fragile, Zingiber fraseri, Zingiber georgeae, Zingiber gracile, Zingiber gramineum, Zingiber griffithii, Zingiber guangxiense, Zingiber gulinense, Zingiber idea, Zingiber incomptum, Zingiber inflexum, Zingiber integrilabrum, Zingiber integrum, Zingiber intermedium, Zingiber isanense, Zingiber junceum, Zingiber kawagoii, Zingiber kelabitianum, Zingiber kerrii, Zingiber kunstleri, Zingiber lambii, Zingiber laoticum, Zingiber larsenii, Zingiber latifolium, Zingiber leptorrhizum, Zingiber leptostachyum, Zingiber ligulatum, Zingiber lingyunense, Zingiber loerzingii, Zingiber longibracteatum, Zingiber longiglande, Zingiber longiligulatum, Zingiber longipedunculatum, Zingiber longyanjiang, Zingiber macradenium, Zingiber macrocephalum, Zingiber macroglossum, Zingiber macrorrhynchus, Zingiber malaysianum, Zingiber marginatum, Zingiber martini, Zingiber matutumense, Zingiber meghalayense, Zingiber mekongense, Zingiber menghaiense, Zingiber mioga, Zingiber mole, Zingiber monglaense, Zingiber monophylum, Zingiber montanum, Zingiber multibracteatum, Zingiber neesanum, Zingiber neglectum, Zingiber negrosense, Zingiber neotruncatum, Zingiber newmanii, Zingiber nigrimaculatum, Zingiber nimmonii, Zingiber niveum, Zingiber nudicarpum, Zingiber odoriferum, Zingiber officinale, Zingiber oligophyllum, Zingiber olivaceum, Zingiber orbiculatum, Zingiber ottensii, Zingiber pachysiphon, Zingiber panduratum, Zingiber papuanum, Zingiber pardocheilum, Zingiber parishii, Zingiber paucipunctatum, Zingiber pellitum, Zingiber pendulum, Zingiber peninsulare, Zingiber petiolatum, Zingiber phillippsiae, Zingiber phumiangense, Zingiber pleiostachyum, Zingiber porphyrosphaerum, Zingiber pseudopungens, Zingiber puberulum, Zingiber pubisquama, Zingiber pyroglossum, Zingiber raja, Zingiber recurvatum, Zingiber roseum, Zingiber rubens, Zingiber rufopilosum, Zingiber sirindhorniae, Zingiber simaoense, Zingiber smilesianum, Zingiber spectabile, Zingiber squarrosum, Zingiber stenostachys, Zingiber stipitatum, Zingiber striolatum, Zingiber sulphureum, Zingiber tenuiscapus, Zingiber thorelii, Zingiber tuanjuum, Zingiber vanlithianum, Zingiber vittacheilum, Zingiber velutinum, Zingiber vinosum, Zingiber viridiflavum, Zingiber wandingense, Zingiber wightianum, Zingiber wrayi, Zingiber yingjiangense, Zingiber yunnanense, Zingiber sadakornii, and Zingiber zerumbet.

In some embodiments, the Zingiber extract is an extract of Zingiber officinale. In other embodiments, the Zingiber extract is an extract of Zingiber officinale root or blub.

The genus Cinnamomum is a genus of evergreen aromatic trees and shrubs belonging to the laurel family, which includes, inter alia, the species Cinnamomum acuminatifolium, Cinnamomum acuminatissimum, Cinnamomum acutatum, Cinnamomum africanum, Cinnamomum aggregatum, Cinnamomum alainii, Cinnamomum alatum, Cinnamomum albiflorum, Cinnamomum alcinii, Cinnamomum alexei, Cinnamomum alibertii, Cinnamomum alternifolium, Cinnamomum altissimum, Cinnamomum ammannii, Cinnamomum amoenum, Cinnamomum amplexicaule, Cinnamomum amplifolium, Cinnamomum anacardium, Cinnamomum andersonii, Cinnamomum angustifolium, Cinnamomum angustitepalum, Cinnamomum antillarum, Cinnamomum appelianum, Cinnamomum arbusculum, Cinnamomum archboldianum, Cinnamomm areolatocostae, Cinnamomum areolatum, Cinnamomum arfakense, Cinnamomum argenteum, Cinnamomum arsenei, Cinnamomum asa-grayi, Cinnamomum assamicum, Cinnamomum aubletii, Cinnamomum aureo-fulvum, Cinnamomum austral, Cinnamomum austro-sinense, Cinnamomum austro-yunnanense, Cinnamomum bahianum, Cinnamomum bahiense, Cinnamomum baileyanum, Cinnamomum baillonii, Cinnamomum balansae, Cinnamomum bamoense, Cinnamomum barbato-axillatum, Cinnamomum barbeyanum, Cinnamomum barlowii, Cinnamomum bartheifolium, Cinnamomum barthii, Cinnamomum bazania, Cinnamomum beccarii, Cinnamomum bejolghota, Cinnamomum bengalense, Cinnamomum biafranum, Cinnamomum bintulense, Cinnamomum birmanicum, Cinnamomum blumei, Cinnamomum bodinieri, Cinnamomum bonii, Cinnamomum bonplandii, Cinnamomum borneense, Cinnamomum bourgeauvianum, Cinnamomum boutonii, Cinnamomum brachythyrsum, Cinnamomum bractefoliaceum, Cinnamomum burmannii, Cinnamomum cambodianum, Cinnamomum camphora, Cinnamomum cassia, Cinnamomum caudiferum, Cinnamomum cebuense, Cinnamomum chartophyllum, Cinnamomum citriodorum, Cinnamomum contractum, Cinnamomum culilawan, Cinnamomum dubium, Cinnamomum elegans, Cinnamomum filipes, Cinnamomum glanduliferum, Cinnamomum glaucescens, Cinnamomum ilicioides, Cinnamomum impressinervium, Cinnamomum iners, Cinnamomum japonicum, Cinnamomum javanicum, Cinnamomum jensenianum, Cinnamomum kanehirae, Cinnamomum kotoense, Cinnamomum kwangtungense, Cinnamomum liangii, Cinnamomum longepaniculatum, Cinnamomum longipetiolatum, Cinnamomum loureiroi, Cinnamomum mairei, Cinnamomum malabatrum, Cinnamomum mercadoi, Cinnamomum micranthum, Cinnamomum migao, Cinnamomum mollifolium, Cinnamomum oliveri, Cinnamomum osmophloeum, Cinnamomum ovalifolium, Cinnamomum parthenoxylon, Cinnamomum pauciflorum, Cinnamomum pedunculatum, Cinnamomum philippinense, Cinnamomum pingbienense, Cinnamomum pittosporoides, Cinnamomum platyphyllum, Cinnamomum porphyrium, Cinnamomum porrectum, Cinnamomum reticulatum, Cinnamomum rigidissimum, Cinnamomum saxatile, Cinnamomum septentrionale, Cinnamomum sinharajaense, Cinnamomum sintoc, Cinnamomum subavenium, Cinnamomum tamala, Cinnamomum tenuipilum, Cinnamomum tonkinense, Cinnamomum triplinerve, Cinnamomum tsangii, Cinnamomum validinerve, Cinnamomum verum, Cinnamomum virens, Cinnamomum walaiwarense, and Cinnamomum wilsonii.

In some embodiments, the Cinnamomum extract is an extract of Cinnamomum cassia. In other embodiments, the Cinnamomum extract is an extract of Cinnamomum cassia bark or twig.

The genus Paeonia is a group of flowering plants in the family Paeoniaceae, which includes, inter alia, the species Paeonia algeriensis, Paeonia anomala, Paeonia arietina, Paeonia broteri, Paeonia brownii, Paeonia californica, Paeonia cambessedesii, Paeonia clusii, Paeonia coriacea, Paeonia corsica, Paeonia daurica, Paeonia emodi, Paeonia intermedia, Paeonia kesrouanensis, Paeonia lactiflora, Paeonia mairei, Paeonia mascula, Paeonia obovate, Paeonia officinalis, Paeonia parnassica, Paeonia peregrina, Paeonia sterniana, Paeonia tenuifolia, Paeonia veitchii, Paeonia decomposita, Paeonia delavayi, Paeonia jishanensis, Paeonia ludlowii, Paeonia ostia, Paeonia qiui, and Paeonia rockii.

In some embodiments, the Paeonia extract is an extract of Paeonia lactiflora. In other embodiments, the Paeonia extract is an extract of Paeonia lactiflora root.

The genus Terminalia is a genus of large trees of the flowering plant family Combretaceae, which includes, inter alia, the species Terminalia acuminata, Terminalia albida, Terminalia altissima, Terminalia amazonia, Terminalia arbuscula, Terminalia archipelagi, Terminalia arenicola, Terminalia argentea, Terminalia arjuna, Terminalia australis, Terminalia avicennioides, Terminalia bellerica (Myrobalanus bellerica), Terminalia bentzoe, Terminalia bialata, Terminalia brachystemma, Terminalia brassii, Terminalia brownii, Terminalia bucidoides, Terminalia buceras, Terminalia bursarina, Terminalia calamansanai, Terminalia carpentariae, Terminalia catappa, Terminalia chebula, Terminalia cherrieri, Terminalia ciliata, Terminalia citrina, Terminalia corticosa, Terminalia eddowesii, Terminalia elliptica, Terminalia eriostachya, Terminalia ferdinandiana, Terminalia foetidissima, Terminalia franchetii, Terminalia glabrescens, Terminalia glaucifolia, Terminalia grandiflora, Terminalia hararensis, Terminalia hecistocarpa, Terminalia intermedia, Terminalia ivorensis, Terminalia januariensis, Terminalia kaernbachii, Terminalia kangeanensis, Terminalia kuhlmannii, Terminalia latifolia, Terminalia latipes, Terminalia littoralis, Terminalia macroptera, Terminalia mantaly, Terminalia microcarpa, Terminalia muelleri, Terminalia myriocarpa, Terminalia nitens, Terminalia novocaledonica, Terminalia oblonga, Terminalia oblongata, Terminalia obovata, Terminalia oliveri, Terminalia paniculata, Terminalia parviflora, Terminalia pellucida, Terminalia petiolaris, Terminalia phanerophlebia, Terminalia phellocarpa, Terminalia porphyrocarpa, Terminalia procera, Terminalia prunioides, Terminalia reitzii, Terminalia rerei, Terminalia richii, Terminalia saffordii, Terminalia schimperiana, Terminalia sericea, Terminalia sericocarpa, Terminalia subspathulata, Terminalia superba, Terminalia triflora, Terminalia trifoliata, and Terminalia tripteroides.

In some embodiments, the Terminalia extract is an extract of Terminalia chebula or Terminalia ballerica. In other embodiments, the Terminalia extract is an extract of Terminalia ballerica or Terminalia chebula leaves, fruit, pericarp, or a mixture thereof.

The genus Commiphora is a genus of flowering plants in the family Burseraceae, which includes, inter alia, the species Commiphora africana, Commiphora alaticaulis, Commiphora angolensis, Commiphora boranensis, Commiphora caudata, Commiphora ciliata, Commiphora confusa, Commiphora corrugata, Commiphora erosa, Commiphora gileadensis, Commiphora glandulosa, Commiphora guidottii, Commiphora guillauminii, Commiphora habessinica, Commiphora harveyi, Commiphora humbertii, Commiphora kataf, Commiphora kua, Commiphora madagascariensis, Commiphora monoica, Commiphora mossambicensis, Commiphora myrrha, Commiphora saxicola, Commiphora schimperi, Commiphora simplicifolia, Commiphora sphaerocarpa, Commiphora stocksiana, Commiphora unilobata, and Commiphora wightii.

In some embodiments, the Commiphora extract is an extract of Commiphora myrrha. In other embodiments, the Commiphora extract is an extract of Commiphora myrrha resin.

The genus Boswellia is another genus of flowering plants in the family Burseraceae, which includes, inter alia, the species Boswellia ameero, Boswellia boranensis, Boswellia bricchettii, Boswellia bullata, Boswellia chariensis, Boswellia dalzielii, Boswellia dioscoridis, Boswellia elegans, Boswellia elongate, Boswellia frereana, Boswellia globosa, Boswellia hildebrandtii, Boswellia holstii, Boswellia madagascariensis, Boswellia microphylla, Boswellia multifoliolate, Boswellia nana, Boswellia neglecta, Boswellia odorata, Boswellia ogadensis, Boswellia ovalifoliolata, Boswellia papyrifera, Boswellia pirottae, Boswellia popoviana, Boswellia rivae, Boswellia ruspoliana, Boswellia sacra, Boswellia serrata, and Boswellia socotrana.

In some embodiments, the Boswellia extract is an extract of Boswellia serrata. In other embodiments, the Boswellia extract is an extract of Boswellia serrata resin.

The genus Dipterocarpus is another genus of plants in the family Dipterocarpaceae, which includes, inter alia, the species Dipterocarpus acutangulus, Dipterocarpus alatus, Dipterocarpus applanatus, Dipterocarpus baudii, Dipterocarpus borneensis, Dipterocarpus bourdilloni, Dipterocarpus caudatus, Dipterocarpus caudiferus, Dipterocarpus chartaceus, Dipterocarpus cinereus, Dipterocarpus concavus, Dipterocarpus condorensis, Dipterocarpus confertus, Dipterocarpus conformis, Dipterocarpus coriaceus, Dipterocarpus cornutus, Dipterocarpus costatus, Dipterocarpus costulatus, Dipterocarpus crinitus, Dipterocarpus cuspidatus, Dipterocarpus dyeri, Dipterocarpus elongatus, Dipterocarpus eurynchus, Dipterocarpus fagineus, Dipterocarpus fusiformis, Dipterocarpus geniculatus, Dipterocarpus glabrigemmatus, Dipterocarpus glandulosus, Dipterocarpus globosus, Dipterocarpus gracilis, Dipterocarpus grandiflorus, Dipterocarpus hasseltii, Dipterocarpus hispidus, Dipterocarpus humeratus, Dipterocarpus indicus, Dipterocarpus insignis, Dipterocarpus intricatus, Dipterocarpus kerrii, Dipterocarpus kunstleri, Dipterocarpus lamellatus, Dipterocarpus littoralis, Dipterocarpus lowii, Dipterocarpus megacarpus, Dipterocarpus mundus, Dipterocarpus nudus, Dipterocarpus oblongifolius, Dipterocarpus obtusifolius, Dipterocarpus ochraceus, Dipterocarpus orbicularis, Dipterocarpus pachyphyllus, Dipterocarpus palembanicus, Dipterocarpus perakensis, Dipterocarpud pseudocornutus, Dipterocarpus retusus, Dipterocarpus rigidus, Dipterocarpus rotundifolius, Dipterocarpus sarawakensis, Dipterocarpus semivestitus, Dipterocarpus stellatus, Dipterocarpus sublamellatus, Dipterocarpus tempehes, Dipterocarpus tuberculatus, Dipterocarpus turbinatus, Dipterocarpus Validus, Dipterocarpus verrucosus, and Dipterocarpus zeylanicus.

In some embodiments, the Dipterocarpus extract is an extract of Dipterocarpus turbinatus. In other embodiments, the Dipterocarpus extract is an extract of Dipterocarpus turbinatus resin.

In some embodiments, the Copaiba extract is an extract of Copaiba langsdorffii. In other embodiments, the Copaiba extract is an extract of Copaiba langsdorffii resin or bark.

The genus Malpighia is a genus of flowing plants in the family Malpighiaceae, which includes, inter alia, the species Malpighia aquifolia, Malpighia cauliflora, Malpighia coccigera, Malpighia cubensis, Malpighia emarginata, Malpighia fucata, Malpighia glabra, Malpighia harrisii, Malpighia mexicana, Malpighia obtusifolia, Malpighia polytricha, Malpighia proctorii, Malpighia setosa, Malpighia suberosa, and Malpighia urens.

In some embodiments, the Malpighia extract is an extract of Malpighia glabra or Malpighia emarginata. In other embodiments, the Malpighia extract is an extract of Malpighia glabra or Malpighia emarginata fruit.

The genus Thymus is a group of plants in the family Lamiaceae, which includes, inter alia, the species Thymus adamovicii, Thymus altaicus, Thymus amurensis, Thymus boissieri, Thymus bracteosus, Thymus broussonetii, Thymus caespititius, Thymus camphoratus, Thymus capitatus, Thymus capitellatus, Thymus camphoratus, Thymus carnosus, Thymus cephalotus, Thymus cherlerioides, Thymus ciliatus, Thymus cilicicus, Thymus cimicinus, Thymus citriodorus, Thymus comosus, Thymus comptus, Thymus curtus, Thymus decussatus, Thymus disjunctus, Thymus doerfleri, Thymus glabrescens, Thymus herba-barona, Thymus hirsutus, Thymus hyemalis, Thymus inaequalis, Thymus integer, Thymus lanuginosus, Thymus leucospermus, Thymus leucotrichus, Thymus longicaulis, Thymus longiflorus, Thymus mandschuricus, Thymus marschallianus, Thymus mastichina, Thymus membranaceus, Thymus mongolicus, Thymus moroderi, Thymus nervulosus, Thymus nummularis, Thymus odoratissimus, Thymus pallasianus, Thymus pallidus, Thymus pannonicus, Thymus praecox, Thymus proximus, Thymus pseudolanuginosus, Thymus pulegioides, Thymus quinquecostatus, Thymus richardii, Thymus satureioides, Thymus serpyllum, Thymus sibthorpii, Thymus striatus, Thymus thracicus, Thymus villosus, Thymus vulgaris and Thymus zygis.

In some embodiments, the Thymus extract is an extract of Thymus vulgaris. In other embodiments, the Thymus extract is an extract of Thymus vulgaris leaves.

The genus Origanum is another herbaceous perennials group of plants in the family Lamiaceae, which includes, inter alia, the species Origanum acutidens, Origanum adanense, Origanum adonidis, Origanum akhdarense, Origanum amanum, Origanum barbarae, Origanum bargyli, Origanum bilgeri, Origanum boissieri, Origanum calcaratum, Origanum compactum, Origanum cordifolium, Origanum cyrenaicum, Origanum dayi, Origanum Dictamnus, Origanum dolichosiphon, Origanum ehrenbergii, Origanum elongatum, Origanum floribundum, Origanum haradjanii, Origanum haussknechtii, Origanum husnucan-baseri, Origanum hypericifolium, Origanum intercedens, Origanum intermedium, Origanum isthmicum, Origanum jordanicum, Origanum laevigatum, Origanum leptocladum, Origanum libanoticum, Origanum majorana, Origanum lirium, Origanum majoricum, Origanum microphyllum, Origanum minoanum, Origanum minutiflorum, Origanum munzurense, Origanum nebrodense, Origanum onites, Origanum pabotii, Origanum pampaninii, Origanum petraeum, Origanum punonense, Origanum ramonense, Origanum rotundifolium, Origanum saccatum, Origanum scabrum, Origanum sipyleum, Origanum solymicum, Origanum symes, Origanum syriacum, Origanum vetteri, Origanum vogelii, and Origanum vulgare.

In some embodiments, the Origanum extract is an extract of Origanum vulgare. In other embodiments, the Origanum extract is an extract of Origanum vulgare leaves.

The genus Cymbopogon is a group of plants in the family Poaceae, which includes, inter alia, the species Cymbopogon ambiguous, Cymbopogon annamensis, Cymbopogon bhutanicus, Cymbopogon bombycinus, Cymbopogon caesius, Cymbopogon calcicole, Cymbopogon calciphilus, Cymbopogon cambogiensis, Cymbopogon citratus, Cymbopogon clandestinus, Cymbopogon coloratus, Cymbopogon commutatus, Cymbopogon densiflorus, Cymbopogon dependens, Cymbopogon dieterlenii, Cymbopogon distans, Cymbopogon exsertus, Cymbopogon flexuosus, Cymbopogon gidarba, Cymbopogon giganteus, Cymbopogon globosus, Cymbopogon goeringii, Cymbopogon gratus, Cymbopogon jwarancusa, Cymbopogon khasianus, Cymbopogon liangshanensis, Cymbopogon mandalaiaensis, Cymbopogon marginatus, Cymbopogon martini, Cymbopogon mekongensis, Cymbopogon microstachys, Cymbopogon microthecus, Cymbopogon minor, Cymbopogon minutiflorus, Cymbopogon nardus, Cymbopogon nervatus, Cymbopogon obtectus, Cymbopogon osmastonii, Cymbopogon pendulus, Cymbopogon polyneuros, Cymbopogon pospischilii, Cymbopogon procerus, Cymbopogon pruinosus, Cymbopogon queenslandicus, Cymbopogon quinhonensis, Cymbopogon rectus, Cymbopogon refractus, Cymbopogon schoenanthus, Cymbopogon tortilis, Cymbopogon tungmaiensis, Cymbopogon winterianus, and Cymbopogon xichangensis.

In some embodiments, the Cymbopogon extract is an extract of Cymbopogon citratus. In other embodiments, the Anethum extract is an extract of Cymbopogon citratus leaves.

In some embodiments, the Anethum extract is an extract of Anethum graveolens. In other embodiments, the Anethum extract is an extract of Anethum graveolens seeds.

The genus Syzygium is a group of flowering plants in the family Myrtaceae, which includes, inter alia, the species Syzygium alliiligneum, Syzygium amplifolium, Syzygium andamanicum, Syzygium anisatum, Syzygium anisosepalum, Syzygium angophoroides, Syzygium antisepticum, Syzygium aqueum, Syzygium aromaticum, Syzygium austral, Syzygium beddomei, Syzygium bourdillonii, Syzygium canicortex, Syzygium caryophyllatum, Syzygium chanlos, Syzygium chavaran, Syzygium cinereum, Syzygium conglomeratum, Syzygium contractum, Syzygium cordatum, Syzygium cordifolium, Syzygium cormiflorum, Syzygium corynanthum, Syzygium corynocarpa, Syzygium courtallense, Syzygium crebrinerve, Syzygium cumini, Syzygium curranii, Syzygium densiflorum, Syzygium diffusum, Syzygium discophorum, Syzygium duthieanum, Syzygium dyerianum, Syzygium elegans, Syzygium erythrocalyx, Syzygium eucalyptoides, Syzygium fergusonii, Syzygium fibrosum, Syzygium fijiense, Syzygium flosculiferum, Syzygium forte, Syzygium formosanum, Syzygium francisii, Syzygium fullagarii, Syzygium glaucum, Syzygium gracilipes, Syzygium graeme-andersoniae, Syzygium grande, Syzygium guehoi, Syzygium gustavioides, Syzygium guineense, Syzygium hodgkinsoniae, Syzygium jambos, Syzygium jasminifolium, Syzygium kemamanense, Syzygium kiahii, Syzygium koordersianum, Syzygium kuranda, Syzygium leucoxylon, Syzygium luehmannii, Syzygium maire, Syzygium makul, Syzygium malaccense, Syzygium manii, Syzygium maingayi, Syzygium megacarpum, Syzygium micranthum, Syzygium microphyllum, Syzygium minus, Syzygium monimioides, Syzygium moorei, Syzygium myhendrae, Syzygium neesianum, Syzygium nemestrinum, Syzygium nervosum, Syzygium oblatum, Syzygium occidentale, Syzygium oleosum, Syzygium oliganthum, Syzygium oreophilum, Syzygium palghatense, Syzygium paniculatum, Syzygium parameswaranii, Syzygium patentinerve, Syzygium papyraceum, Syzygium pauper, Syzygium pendens, Syzygium perakense, Syzygium pergamentaceum, Syzygium phaeophyllum, Syzygium politum, Syzygium polyanthum, Syzygium pondoense, Syzygium praineanum, Syzygium pseudofastigiatum, Syzygium purpureum, Syzygium quadrangulatum, Syzygium quadribracteatum, Syzygium ramavarmae, Syzygium revolutum, Syzygium richii, Syzygium samarangense, Syzygium sandwicensis, Syzygium smithii, Syzygium suborbiculare, Syzygium syzygioides, and Syzygium travancoricum.

In some embodiments, the Syzygium extract is an extract of Syzygium aromaticum. In other embodiments, the Syzygium extract is an extract of Syzygium aromaticum flowers or buds.

The genus Magnolia is a group of flowering plants in the family Magnoliaceae, which includes, inter alia, the species Magnolia acuminata, Magnolia alba, Magnolia albosericea, Magnolia allenii, Magnolia amazonica, Magnolia amoena, Magnolia angatensis, Magnolia angustioblonga, Magnolia annamensis, Magnolia arcabucoana, Magnolia argyrothricha, Magnolia aromatica, Magnolia ashtonii, Magnolia baillonii, Magnolia balansae, Magnolia banghamii, Magnolia bankardiorum, Magnolia bawangensis, Magnolia betongensis, Magnolia bintuluensis, Magnolia biondii, Magnolia blaoensis, Magnolia blumei, Magnolia boliviana, Magnolia borneensis, Magnolia braianensis, Magnolia calimaensis, Magnolia calophylla, Magnolia calophylloides, Magnolia campbellii, Magnolia cararensis, Magnolia caricifragrans, Magnolia carsonii, Magnolia cathcartii, Magnolia cavaleriei, Magnolia caveana, Magnolia cespedesii, Magnolia champaca, Magnolia championii, Magnolia changhuntana, Magnolia chapensis, Magnolia chevalieri, Magnolia chimantensis, Magnolia chocoensis, Magnolia citrata, Magnolia clemensiorum, Magnolia coco, Magnolia colombiana, Magnolia compressa, Magnolia conifera, Magnolia coriacea, Magnolia coronata, Magnolia crassipes, Magnolia cristalensis, Magnolia cubensis, Magnolia cylindrica, Magnolia dandyi, Magnolia dawsoniana, Magnolia decidua, Magnolia delavayi, Magnolia denudate, Magnolia dixonii, Magnolia dodecapetala, Magnolia doltsopa, Magnolia domingensis, Magnolia duclouxii, Magnolia duperreana, Magnolia ekmannii, Magnolia elegans, Magnolia elegantifolia, Magnolia emarginata, Magnolia ernestii, Magnolia espinalii, Magnolia figlarii, Magnolia figo, Magnolia fistulosa, Magnolia flaviflora, Magnolia floribunda, Magnolia fordiana, Magnolia foveolate, Magnolia fraseri, Magnolia fujianensis, Magnolia fulva, Magnolia garrettii, Magnolia georgii, Magnolia gigantifolia, Magnolia gilbertoi, Magnolia gioi, Magnolia globosa, Magnolia gloriensis, Magnolia grandiflora, Magnolia grandis, Magnolia griffithii, Magnolia guangdongensis, Magnolia guangxiensis, Magnolia guatapensis, Magnolia guatemalensis, Magnolia guatemalensis, Magnolia guerrerensis, Magnolia gustavii, Magnolia hamorii, Magnolia henaoi, Magnolia henryi, Magnolia hernandezii, Magnolia hodgsonii, Magnolia hongheensis, Magnolia hookeri, Magnolia hypolampra, Magnolia iltisiana, Magnolia insignis, Magnolia irwiniana, Magnolia iteophylla, Magnolia jaenensis, Magnolia jardinensis, Magnolia kachirachirai, Magnolia katiorum, Magnolia kingii, Magnolia kisopa, Magnolia kobus, Magnolia koordersiana, Magnolia krusei, Magnolia kwangsiensis, Magnolia kwangtungensis, Magnolia lacandonica, Magnolia lacei, Magnolia laevifolia, Magnolia lanuginosoides, Magnolia lasia, Magnolia lanuginosa, Magnolia lenticellata, Magnolia leveilleana, Magnolia liliifera, Magnolia liliiflora, Magnolia loebneri, Magnolia longipedunculata, Magnolia lotungensis, Magnolia lucida, Magnolia macclurei, Magnolia macklottii, Magnolia macrophylla, Magnolia magnifolia, Magnolia mahechae, Magnolia manguillo, Magnolia mannii, Magnolia mariusjacobsia, Magnolia martini, Magnolia masticata, Magnolia maudiae, Magnolia mediocris, Magnolia Mexicana, Magnolia microcarpa, Magnolia microtricha, Magnolia minor, Magnolia montana, Magnolia morii, Magnolia nana, Magnolia narinensis, Magnolia neillii, Magnolia nilagirica, Magnolia nitida, Magnolia oblonga, Magnolia obovalifolia, Magnolia obovata, Magnolia odora, Magnolia odoratissima, Magnolia officinalis, Magnolia omeiensis, Magnolia opipara, Magnolia ovata, Magnolia ovoidea, Magnolia pacifica, Magnolia pahangensis, Magnolia pallescens, Magnolia panamensis, Magnolia patungensis, Magnolia persuaveolens, Magnolia philippinensis, Magnolia pealiana, Magnolia platyphylla, Magnolia pleiocarpa, Magnolia poasana, Magnolia polyhypsophylla, Magnolia portoricensis, Magnolia praecalva, Magnolia ptaritepuiana, Magnolia pterocarpan, Magnolia pubescens, Magnolia punduana, Magnolia quetzal, Magnolia rabaniana, Magnolia rajaniana, Magnolia rimachii, Magnolia rostrata, Magnolia rufibarbata, Magnolia sabahensis, Magnolia salicifolia, Magnolia sapaensis, Magnolia sarawakensis, Magnolia sambuensis, Magnolia santanderiana, Magnolia sargentiana, Magnolia schiedeana, Magnolia scortechinii, Magnolia sellowiana, Magnolia sharpie, Magnolia shiluensis, Magnolia sieboldii, Magnolia silvioi, Magnolia singapurensis, Magnolia sinica, Magnolia sirindhorniae, Magnolia sororum, Magnolia soulangeana, Magnolia sphaerantha, Magnolia splendens, Magnolia sprengeri, Magnolia stellata, Magnolia striatifolia, Magnolia subulifera, Magnolia sulawesiana, Magnolia sumatrae, Magnolia tamaulipana, Magnolia thailandica, Magnolia tripetala, Magnolia tsiampacca, Magnolia urraoense, Magnolia utilis, Magnolia vazquezii, Magnolia venezuelensis, Magnolia ventii, Magnolia villosa, Magnolia virginiana, Magnolia virolinensis, Magnolia vrieseana, Magnolia wilsonii, Magnolia wolfii, Magnolia xanthantha, Magnolia xiana, Magnolia xinganensis, Magnolia yarumalense, Magnolia yoroconte, Magnolia yunnanensis, Magnolia yuyuanensis, Magnolia zenii, and Magnolia zhengyiana.

In some embodiments, the Magnolia extract is an extract of Magnolia officinalis. In other embodiments, the Magnolia extract is an extract of Magnolia officinalis bark.

The genus Pimenta is a group of flowering plants in the myrtle family, which includes, inter alia, the species Pimenta adenoclada, Pimenta berciliae, Pimenta cainitoides, Pimenta dioica (allspice), Pimenta ferruginea, Pimenta filipes, Pimenta guatemalensis, Pimenta haitiensis, Pimenta intermedia, Pimenta jamaicensis, Pimenta obscura, Pimenta odiolens, Pimenta oligantha, Pimenta podocarpoides, Pimenta pseudocaryophyllus, Pimenta racemosa, Pimenta samanensis, Pimenta richardii, and Pimenta yumana.

In some embodiments, the Pimenta extract is an extract of Pimenta dioica. In other embodiments, the Pimenta extract is an extract of Pimenta dioica fruit.

The genus Salvia is a group of flowering plants in the family Lamiaceae, which includes, inter alia, the species Salvia absconditiflora, Salvia acuminata, Salvia adenocaulon, Salvia Adenophora, Salvia adenophylla, Salvia adiantifolia, Salvia adoxoides, Salvia aegyptiaca, Salvia aequidens, Salvia aequidistans, Salvia aerea, Salvia aethiopis, Salvia Africana, Salvia africana-lutea, Salvia alamosana, Salvia alariformis, Salvia alata, Salvia alatipetiolata, Salvia alba, Salvia albicalyx, Salvia albicaulis, Salvia albiflora, Salvia albimaculata, Salvia albocaerulea, Salvia alborosea, Salvia alexeenkoi, Salvia algeriensis, Salvia aliciae, Salvia altissima, Salvia alvajaca, Salvia amethystine, Salvia amissa, Salvia amplexicaulis, Salvia amplicalyx, Salvia amplifrons, Salvia anastomosans, Salvia anatolica, Salvia andreji, Salvia anguicoma, Salvia angulate, Salvia angustiarum, Salvia apiana, Salvia apparicii, Salvia appendiculate, Salvia arabica, Salvia aramiensis, Salvia arborescens, Salvia arbuscula, Salvia arduinervis, Salvia arenaria, Salvia areolate, Salvia argentea, Salvia Ariana, Salvia aridicola, Salvia aristate, Salvia arizonica, Salvia arthrocoma, Salvia articulata, Salvia aspera, Salvia asperata, Salvia asperifolia, Salvia assurgens, Salvia atrocalyx, Salvia atrocyanea, Salvia atropaenulata, Salvia atropatana, Salvia atropurpurea, Salvia atrorubra, Salvia aucheri, Salvia aurita, Salvia austriaca, Salvia austromelissodora, Salvia axillaris, Salvia axilliflora, Salvia ayavacensis, Salvia ayayacensis, Salvia aytachii, Salvia azurea, Salvia bahorucona, Salvia baimaensis, Salvia balansae, Salvia balaustina, Salvia baldshuanica, Salvia ballotiflora, Salvia ballsiana, Salvia bariensis, Salvia barrelieri, Salvia bazmanica, Salvia beckeri, Salvia benthamiana, Salvia betulifolia, Salvia bifidocalyx, Salvia biserrate, Salvia blancoana, Salvia blepharophylla, Salvia boegei, Salvia bogotensis, Salvia booleana, Salvia borjensis, Salvia bowleyana, Salvia brachyantha, Salvia brachyloba, Salvia brachyloma, Salvia brachyodon, Salvia brachyodonta, Salvia brachyphylla, Salvia bracteate, Salvia brandegeei, Salvia breviconnectivata, Salvia breviflora, Salvia brevilabra, Salvia brevipes, Salvia broussonetii, Salvia buchananii, Salvia bucharica, Salvia buchii, Salvia bulleyana, Salvia bullulata, Salvia caaguazuensis, Salvia cabonii, Salvia cabulica, Salvia cacaliifolia, Salvia cadmica, Salvia caespitosa, Salvia calaminthifolia, Salvia calcicole, Salvia californica, Salvia calolophos, Salvia camarifolia, Salvia campanulate, Salvia campicola, Salvia camporum, Salvia campylodonta, Salvia canariensis, Salvia candelabrum, Salvia candicans, Salvia candidissima, Salvia canescens, Salvia capillosa, Salvia carbonoi, Salvia cardenasii, Salvia cardiophylla, Salvia carduacea, Salvia carnea, Salvia cassia, Salvia castanea, Salvia cataractarum, Salvia caudata, Salvia cavaleriei, Salvia caymanensis, Salvia cedronella, Salvia cedrosensis, Salvia ceratophylla, Salvia cerradicola, Salvia chalarothyrsa, Salvia chamaedryoides, Salvia chamelaeagnea, Salvia chanryoenica, Salvia chapadensis, Salvia chapalensis, Salvia chiapensis, Salvia chicamochae, Salvia chienii, Salvia chinensis, Salvia chionantha, Salvia chionopeplica, Salvia chionophylla, Salvia chloroleuca, Salvia chorassanica, Salvia chrysophylla, Salvia chudaei, Salvia chunganensis, Salvia cilicica, Salvia cinica, Salvia cinnabarina, Salvia circinnata, Salvia clarendonensis, Salvia clausa, Salvia clevelandii, Salvia clinopodioides, Salvia coahuilensis, Salvia coccinea, Salvia cocuyana, Salvia codazziana, Salvia coerulea, Salvia cognata, Salvia colonica, Salvia columbariae, Salvia comayaguana, Salvia compar, Salvia compressa, Salvia compsostachys, Salvia concolor, Salvia confertiflora, Salvia congestiflora, Salvia connivens, Salvia consimilis, Salvia consobrina, Salvia corazonica, Salvia cordata, Salvia coriana, Salvia corrugate, Salvia costaricensis, Salvia costata, Salvia coulteri, Salvia crinigera, Salvia crucis, Salvia cruikshanksii, Salvia cryptoclada, Salvia cryptodonta, Salvia cuatrecasana, Salvia cubensis, Salvia curta, Salvia curticalyx, Salvia curtiflora, Salvia curviflora, Salvia cuspidate, Salvia cyanantha, Salvia cyanescens, Salvia cyanicalyx, Salvia cyanocephala, Salvia cyanotropha, Salvia cyclostegia, Salvia cylindriflora, Salvia cynica, Salvia dabieshanensis, Salvia darcyi, Salvia dasyantha, Salvia decumbens, Salvia decurrens, Salvia densiflora, Salvia dentata, Salvia deserta, Salvia deserti, Salvia desoleana, Salvia diamantina, Salvia dichlamys, Salvia dichroantha, Salvia digitaloides, Salvia discolor, Salvia disermas, Salvia disjuncta, Salvia divaricate, Salvia divinorum, Salvia dolichantha, Salvia dolomitica, Salvia dombeyi, Salvia dominica, Salvia dorisiana, Salvia dorrii, Salvia drobovii, Salvia drusica, Salvia drymocharis, Salvia dryophila, Salvia dugesiana, Salvia dumetorum, Salvia durantiflora, Salvia durifolia, Salvia duripes, Salvia ecbatanensis, Salvia ecuadorensis, Salvia eichleriana, Salvia eigii, Salvia eizi, Salvia ekimiana, Salvia elegans, Salvia elenevskyi, Salvia emaciate, Salvia engelmannii, Salvia eplingiana, Salvia eremophila, Salvia eremostachya, Salvia eriocalyx, Salvia eriophora, Salvia ernesti, Salvia erythropoda, Salvia erythrostephana, Salvia erythrostoma, Salvia espirito, Salvia euphratica, Salvia evansiana, Salvia exilis, Salvia expansa, Salvia exserta, Salvia fairuziana, Salvia falcata, Salvia farinacea, Salvia filicifolia, Salvia filifolia, Salvia filipes, Salvia firma, Salvia flaccida, Salvia flaccidifolia, Salvia flava, Salvia flocculosa, Salvia florida, Salvia fluviatilis, Salvia fominii, Salvia formosa, Salvia forreri, Salvia forsskaolei, Salvia foveolate, Salvia fracta, Salvia fragarioides, Salvia freyniana, Salvia frigida, Salvia fruticetorum, Salvia fruticosa, Salvia fruticulosa, Salvia fugax, Salvia fulgens, Salvia funckii, Salvia funerea, Salvia fusca, Salvia fuscomanicata, Salvia garedjii, Salvia gariepensis, Salvia garipensis, Salvia gattefossei, Salvia gesneriiflora, Salvia ghiesbreghtii, Salvia glabra, Salvia glabrata, Salvia glabrescens, Salvia glabricaulis, Salvia glandulifera, Salvia glechomifolia, Salvia glumacea, Salvia glutinosa, Salvia goldmanii, Salvia golneviana, Salvia gontscharowii, Salvia gonzalezii, Salvia gracilipes, Salvia graciliramulosa, Salvia grandifolia, Salvia grandis, Salvia granitica, Salvia gravida, Salvia greatae, Salvia greggii, Salvia grewiifolia, Salvia grisea, Salvia griseifolia, Salvia grossheimii, Salvia guadalujarensis, Salvia guaranitica, Salvia guarinae, Salvia haenkei, Salvia haitiensis, Salvia hajastana, Salvia halaensis, Salvia halophila, Salvia hamulus, Salvia handelii, Salvia hapalophylla, Salvia harleyana, Salvia hasankeyfensis, Salvia hatschbachii, Salvia haussknechtii, Salvia hayatae, Salvia hedgeana, Salvia heerii, Salvia heldreichiana, Salvia helianthemifolia, Salvia henryi, Salvia herbacea, Salvia herbanica, Salvia hermesiana, Salvia herrerae, Salvia heterochroa, Salvia heterofolia, Salvia heterotricha, Salvia hians, Salvia hidalgensis, Salvia hierosolymitana, Salvia hilarii, Salvia hillcoatiae, Salvia himmelbaurii, Salvia hintonii, Salvia hirsute, Salvia hirta, Salvia hirtella, Salvia hispanica, Salvia holwayi, Salvia honania, Salvia hotteana, Salvia huberi, Salvia humboldtiana, Salvia hupehensis, Salvia hydrangea, Salvia hylocharis, Salvia hypargeia, Salvia hypochionaea, Salvia hypoleuca, Salvia incumbens, Salvia incurvata, Salvia indica, Salvia indigocephala, Salvia infuscata, Salvia innoxia, Salvia inornate, Salvia insignis, Salvia insularum, Salvia integrifoli, Salvia interrupta, Salvia intonsa, Salvia involucrate, Salvia iodantha, Salvia iodophylla, Salvia ionocalyx, Salvia isensis, Salvia itaguassuensis, Salvia itatiaiensis, Salvia iuliana, Salvia jacobi, Salvia jaimehintoniana, Salvia jamaicensis, Salvia jaminiana, Salvia jamzadii, Salvia japonica, Salvia jaramilloi, Salvia jorgehintoniana, Salvia Judaica, Salvia jurisicii, Salvia kamelinii, Salvia karabachensis, Salvia karwinskii, Salvia keerlii, Salvia kellermanii, Salvia kermanshahensis, Salvia kiangsiensis, Salvia kiaometiensis, Salvia komarovii, Salvia korolkovii, Salvia koyamae, Salvia kronenburgii, Salvia kurdica, Salvia kuznetzovii, Salvia lachnaioclada, Salvia lachnocalyx, Salvia lachnostachys, Salvia lachnostoma, Salvia laevis, Salvia lamiifolia, Salvia lanceolata, Salvia langlassei, Salvia languidula, Salvia lanicalyx, Salvia lanicaulis, Salvia lanigera, Salvia lankongensis, Salvia lasiantha, Salvia lasiocephala, Salvia Lavandula, Salvia lavandulifolia, Salvia lavanduloides, Salvia laxispicata, Salvia lemmonii, Salvia leninae, Salvia lenta, Salvia leonia, Salvia leptophylla, Salvia leptostachys, Salvia leriifolia, Salvia leucantha, Salvia leucocephala, Salvia leucochlamys, Salvia leucodermis, Salvia leucophylla, Salvia libanensis, Salvia liguliloba, Salvia lilacinocoerulea, Salvia limbate, Salvia lineata, Salvia lipskyi, Salvia littae, Salvia lobbii, Salvia longibracteolata, Salvia longipedicellata, Salvia longispicata, Salvia longistyla, Salvia lophanthoides, Salvia loxensis, Salvia lozanii, Salvia lutescens, Salvia lycioides, Salvia lyrate, Salvia macellaria, Salvia macilenta, Salvia macrocalyx, Salvia macrochlamys, Salvia macrophylla, Salvia macrosiphon, Salvia macrostachya, Salvia madrensis, Salvia mairei, Salvia malvifolia, Salvia manantlanensis, Salvia manaurica, Salvia marashica, Salvia marci, Salvia margaritae, Salvia mattogrossensis, Salvia maximowicziana, Salvia maxonii, Salvia maymanica, Salvia mayorii, Salvia mazatlanensis, Salvia medusa, Salvia meiliensis, Salvia mekongensis, Salvia melaleuca, Salvia melissiflora, Salvia melissodora, Salvia mellifera, Salvia mentiens, Salvia merjamie, Salvia mexiae, Salvia Mexicana, Salvia microdictya, Salvia microphylla, Salvia microstegia, Salvia miltiorrhiza, Salvia minarum, Salvia miniate, Salvia mirzayanii, Salvia misella, Salvia mocinoi, Salvia modesta, Salvia modica, Salvia mohavensis, Salvia monantha, Salvia monclovensis, Salvia moniliformis, Salvia montbretia, Salvia montecristina, Salvia moorcroftiana, Salvia mornicola, Salvia mouretii, Salvia muelleri, Salvia muirii, Salvia mukerjeei, Salvia multicaulis, Salvia munzii, Salvia muscarioides, Salvia namaensis, Salvia nana, Salvia nanchuanensis, Salvia napifolia, Salvia nazalena, Salvia nemoralis, Salvia nemorosa, Salvia neovidensis, Salvia nepetoides, Salvia nervata, Salvia nervosa, Salvia nilotica, Salvia nipponica, Salvia nitida, Salvia nubicola, Salvia nubigena, Salvia nubilorum, Salvia nutans, Salvia nydeggeri, Salvia oaxacana, Salvia oblongifolia, Salvia obtorta, Salvia obtusata, Salvia obumbrate, Salvia occidentalis, Salvia occidua, Salvia occultiflora, Salvia ochrantha, Salvia ocimifolia, Salvia odontochlamys, Salvia officinalis, Salvia oligantha, Salvia oligophylla, Salvia ombrophila, Salvia omeiana, Salvia omerocalyx, Salvia opertiflora, Salvia ophiocephala, Salvia oppositiflora, Salvia orbignaei, Salvia oreopola, Salvia oresbia, Salvia orthostachys, Salvia ovalifolia, Salvia oxyphora, Salvia pachyphylla, Salvia pachypoda, Salvia pachystachya, Salvia palaestina, Salvia palealis, Salvia palifolia, Salvia pallida, Salvia palmeri, Salvia pamplonitana, Salvia pannosa, Salvia pansamalensis, Salvia paohsingensis, Salvia paposana, Salvia paraguariensis, Salvia paramicola, Salvia paramiltiorrhiza, Salvia parciflora, Salvia parryi, Salvia parvifolia, Salvia paryskii, Salvia patens, Salvia pauciflora, Salvia pauciserrata, Salvia paupercula, Salvia pavonii, Salvia penduliflora, Salvia peninsularis, Salvia pennellii, Salvia pentstemonoides, Salvia peratica, Salvia perblanda, Salvia peregrina, Salvia perlonga, Salvia perlucida, Salvia perplicata, Salvia perrieri, Salvia persepolitana, Salvia persicifolia, Salvia personata, Salvia pexa, Salvia peyronii, Salvia phaenostemma, Salvia phlomoides, Salvia piasezkii, Salvia pichinchensis, Salvia pilifera, Salvia pineticola, Salvia pinguifolia, Salvia pinnata, Salvia pisidica, Salvia platycheila, Salvia platyphylla, Salvia plebeian, Salvia plectranthoides, Salvia plumosa, Salvia plurispicata, Salvia poculata, Salvia podadena, Salvia pogonochila, Salvia polystachya, Salvia pomifera, Salvia porphyrocalyx, Salvia potaninii, Salvia potentillifolia, Salvia potus, Salvia praestans, Salvia praeterita, Salvia prasiifolia, Salvia pratensis, Salvia prattii, Salvia prilipkoana, Salvia primuliformis, Salvia pringlei, Salvia prionitis, Salvia procurrens, Salvia propinqua, Salvia prostrates, Salvia protracta, Salvia pruinose, Salvia prunelloides, Salvia prunifolia, Salvia przewalskii, Salvia pseudoincisa, Salvia pseudojaminiana, Salvia pseudomisella, Salvia pseudopallida, Salvia pseudoprivoides, Salvia pseudorosmarinus, Salvia pseudoserotina, Salvia psilantha, Salvia psilostachya, Salvia pterocalyx, Salvia pteroura, Salvia puberula, Salvia pubescens, Salvia pulchella, Salvia punctata, Salvia purpurea, Salvia purpusii, Salvia pusilla, Salvia pygmaea, Salvia qimenensis, Salvia quercetorum, Salvia quezelii, Salvia quitensis, Salvia radula, Salvia ramamoorthyana, Salvia ramose, Salvia ranzaniana, Salvia raveniana, Salvia raymondii, Salvia rechingeri, Salvia recognita, Salvia recurve, Salvia reeseana, Salvia reflexa, Salvia regla, Salvia regnelliana, Salvia reitzii, Salvia remota, Salvia repens, Salvia reptans, Salvia retinervia, Salvia reuteriana, Salvia revoluta, Salvia rhodostephana, Salvia rhombifolia, Salvia rhyacophila, Salvia rhytidea, Salvia ringens, Salvia rivularis, Salvia roborowskii, Salvia roemeriana, Salvia roscida, Salvia rosei, Salvia rosifolia, Salvia rosmarinoides, Salvia rostellata, Salvia rubescens, Salvia rubifolia, Salvia rubrifaux, Salvia rubriflora, Salvia rubropunctata, Salvia rufula, Salvia runcinate, Salvia rupicola, Salvia rusbyi, Salvia russellii, Salvia rypara, Salvia rzedowskii, Salvia saccardiana, Salvia saccifera, Salvia sacculus, Salvia sagittate, Salvia sahendica, Salvia salicifolia, Salvia samuelssonii, Salvia sanctae-luciae, Salvia santolinifolia, Salvia sapinea, Salvia sarmentosa, Salvia Saxicola, Salvia scabiosifolia, Salvia scabra, Salvia scabrata, Salvia scabrida, Salvia scandens, Salvia scapiformis, Salvia scapose, Salvia schimperi, Salvia schizocalyx, Salvia schizochila, Salvia schlechteri, Salvia schmalbausenii, Salvia sciaphila, Salvia sclarea, Salvia sclareoides, Salvia sclareopsis, Salvia scoparia, Salvia scutellarioides, Salvia scytinophylla, Salvia secunda, Salvia seemannii, Salvia segtinophylla, Salvia selguapensis, Salvia selleana, Salvia sellowiana, Salvia semiatrata, Salvia seravschanica, Salvia sericeotomentosa, Salvia serotina, Salvia serpyllifolia, Salvia serranoae, Salvia sessei, Salvia sessilifolia, Salvia setosa, Salvia setulose, Salvia shannonii, Salvia sharifii, Salvia sharpie, Salvia sibthorpii, Salvia sigchosica, Salvia sikkimensis, Salvia silvarum, Salvia similis, Salvia sinaloensis, Salvia sinica, Salvia smithii, Salvia smyrnaea, Salvia somalensis, Salvia sonchifolia, Salvia sonklarii, Salvia sonomensis, Salvia sophrona, Salvia sordida, Salvia sparsiflora, Salvia spathacea, Salvia speciosa, Salvia speirematoides, Salvia sphacelifolia, Salvia sphacelioides, Salvia spinosa, Salvia splendens, Salvia sprucei, Salvia squalens, Salvia stachydifolia, Salvia stachyoides, Salvia staminea, Salvia stenophylla, Salvia stibalii, Salvia stolonifera, Salvia striata, Salvia strobilanthoidea, Salvia styphelos, Salvia subaequalis, Salvia subglabra, Salvia subhastata, Salvia subincisa, Salvia submutica, Salvia subobscura, Salvia subpalmatinervis, Salvia subpatens, Salvia subrotunda, Salvia subrubens, Salvia subscandens, Salvia substolonifera, Salvia sucrensis, Salvia suffruticosa, Salvia summa, Salvia superba, Salvia synodonta, Salvia syriaca, Salvia tafallae, Salvia taraxacifolia, Salvia tchihatcheffii, Salvia tebesana, Salvia teddii, Salvia Tehuacana, Salvia tenella, Salvia tenuiflora, Salvia tepicensis, Salvia teresae, Salvia tesquicola, Salvia tetrodonta, Salvia texana, Salvia thermarum, Salvia thomasiana, Salvia thormannii, Salvia thymoides, Salvia thyrsiflora, Salvia tianschanica, Salvia tigrine, Salvia tiliifolia, Salvia tingitana, Salvia tobeyi, Salvia tolimensis, Salvia tomentella, Salvia tomentosa, Salvia tonalensis, Salvia topiensis, Salvia tortuensis, Salvia tortuosa, Salvia townsendii, Salvia trachyphylla, Salvia transhimalaica, Salvia transsylvanica, Salvia trautvetteri, Salvia triangularis, Salvia trichocalycina, Salvia trichoclada, Salvia trichopes, Salvia trichostephana, Salvia tricuspidate, Salvia tricuspis, Salvia trifilis, Salvia trijuga, Salvia tubifera, Salvia tubiflora, Salvia tubulosa, Salvia tuerckheimii, Salvia turcomanica, Salvia turdi, Salvia turneri, Salvia tuxtlensis, Salvia tysonii, Salvia uliginosa, Salvia umbratical, Salvia umbraticola, Salvia umbratilis, Salvia uncinate, Salvia unguella, Salvia unicostate, Salvia univerticillata, Salvia uribei, Salvia urica, Salvia urmiensis, Salvia urolepis, Salvia urticifolia, Salvia uruapana, Salvia valentina, Salvia vargasii, Salvia vaseyi, Salvia vasta, Salvia veneris, Salvia venulosa, Salvia verapazana, Salvia verbascifolia, Salvia verbenaca, Salvia verecunda, Salvia vergeduzica, Salvia vermifolia, Salvia veronicifolia, Salvia verticillate, Salvia vestita, Salvia vialis, Salvia villosa, Salvia virgata, Salvia viridis, Salvia viscida, Salvia viscidifolia, Salvia viscosa, Salvia vitifolia, Salvia vvedenskii, Salvia wagneriana, Salvia wardii, Salvia warszewicziana, Salvia weberbaueri, Salvia weihaiensis, Salvia wendelboi, Salvia whitefoordiae, Salvia whitehousei, Salvia wiedemannii, Salvia willeana, Salvia xalapensis, Salvia xanthocheila, Salvia xanthophylla, Salvia xanthotricha, Salvia xeropapillosa, Salvia yosgadensis, Salvia yukoyukparum, Salvia yunnanensis, and Salvia zacualpanensis.

In some embodiments, the Salvia extract is an extract of Salvia officinalis. In other embodiments, the Salvia extract is an extract of Salvia officinalis leaves.

In some embodiments, the additional plant extract can be selected from Styrax paralleloneurus resin and/or bark extract, Styrax tonkinensis resin and/or bark extract, Styrax tolu resin and/or bark extract, Myroxylon balsamum resin and/or bark extract, Myroxylon peruiferum resin and/or bark extract, Myrocarpus fastigiatus resin and/or bark extract, Nigella sativa seeds extract, Cuminum cyminum seeds extract, Zingiber officinale root or bulb extract, Cinnamomum cassia bark or twig extract, Paeonia lactiflora root extract, Terminalia ballerica or Terminalia chebula leaves, fruit, or pericarp extract, Commiphora myrrha resin extract, Boswellia serrata resin extract, Dipterocarpus turbinatus resin extract, Copaiba langsdorffii resin or bark extract, Malpighia glabra fruit extract, Thymus vulgaris leaves extract, Origanum vulgare leaves extract, Cymbopogon citratus leaves extract, Anethum graveolens seeds extract, Syzygium aromaticum flowers or buds extract, Magnolia officinalis bark extract, Malpighia emarginate fruit extract, Malpighia glabra fruit extract, Pimenta dioica fruit extract, Salvia officinalis leaves extract and mixtures thereof.

According to some embodiments, the at least one plant extract is a saponin material extract obtained from extraction of a one or more plant materials.

Saponin material, as used herein is at least one naturally obtained saponin compound, as known in the art. When isolated from a natural source, the saponin material may be used in its substantially pure form (namely at least 85%, 87%, 92%, 95%, or 98% purity), or may be used as a “saponin-containing extract (also referred to herein for the purpose of brevity as “saponin extract”) isolated by a method known in the art.

The saponin-containing extract, by some embodiments, contains at least between 0.2% and 95 wt % saponins, out of the total weight of the dry content of the extract. In some embodiments, the extract used in accordance with the present disclosure comprises between 0.2% and 99 wt % saponins out of the total weight of the dry content of the extract.

In some embodiments, the saponin extract may comprise between about 10% and about 80 wt % saponins out of the total weight of the dry content of the extract. In other embodiments, the saponin extract may comprise between about 10% and about 60 wt % saponins, between about 10% and about 50 wt % saponins, between about 10% and about 40 wt % saponins, between about 10% and about 30 wt % saponins, or even between about 10% and about 20 wt % saponins out of the total weight of the dry content of the extract. In some embodiments, the saponin extract comprises between about 0.2% and about 10 wt % saponins out of the total weight of the dry content of the extract.

When isolated from a natural source, the saponin extract may be used in its substantially pure form (namely at least 85%, 87%, 92%, 95%, or 98% purity).

The saponin-containing extract may be obtained from any plant material known to comprise saponins. In some embodiments, the saponin material is obtained by extraction from plant material by employing a solvent, water, alcohol or a water/alcohol solution. In some embodiments, the alcohol is ethanol or methanol.

The saponin-containing plant material may be selected from shikakai, soyabeans, beans, peas (Pisum sativum), lucerne, tea, spinach, sugar beet, quinoa, liquorice, sunflower, horse chestnut, ginseng, oats, capsicum peppers, aubergine, tomato seed, alliums, asparagus, yam, fenugreek, yucca and ginseng, lucerne, mung beans, Bupleurum falcatum, Camellia oleifera, Camellia sinensis, Desmodium adscendens, Gypsophila, Panax quinqufolius, Panax japonicas, Quillaja saponaria, Sapindus delavayi, Sapindus mukorossi, Sapindus marginatus, Sapindus saponaria, Sapindus trifoliatus, Saponaria officinalis, and Yucca schidigera or any mixture thereof.

In some embodiments, the saponin extract is obtained from a plant source selected from Camellia oleifera, Camellia sinensis, Quillaja saponaria, Sapindus mukorossi, Sapindus saponaria, and Saponaria officinalis or any mixture thereof. In other embodiments, the saponin extract is obtained from Camellia oleifera, Quillaja saponaria and/or Sapindus mukorossi. Saponin containing material may be purified by any means known in the art, including filtration, centrifugation, re-crystallization, distillation, adsorption, chromatographic methods, fractionation, etc.

The saponin extract may be obtained from any part of the plant, including leaves, stems, roots, bulbs, blossom and fruit (including the skin, flesh and seed of the fruit). In some embodiments, the extracts are obtained from the pericarp of Sapindus mukorossi, or the seed meal of Camellia oleifera.

The compositions of this disclosure may further include one or more functional additives, which may, by some embodiments, be selected from citric acid, and shikimic acid.

In another one of its aspects, the disclosure provides a composition comprising maltol, and at least one plant extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material, and any mixtures or combinations thereof.

By another aspect, the disclosure provides an antimicrobial composition comprising maltol, and at least one plant extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material, and any mixtures or combinations thereof.

Yet in another one of its aspects, the disclosure provides an antimicrobial composition comprising kojic acid, and at least one plant extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material, and any mixtures or combinations thereof.

Yet in a further one of its aspects, the disclosure provides an antimicrobial composition comprising 2-ethyl-3-hydroxy-4H-pyran-4-one, and an extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia and from a saponin-containing plant material, and any mixtures or combinations thereof.

In another aspect, there is provided a composition comprising at antimicrobial effective amount of a mixture of agents, the mixture of agents consisting of at least one 4-pyrone derivative (e.g. maltol) and at least one plant extract obtained from at least one plant of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material, and any mixtures or combinations thereof.

Yet a further aspect provides for an add-on antimicrobial composition comprising a combination of at least 4-pyrone derivative (e.g. maltol) and at least one plant extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material, and any mixtures or combinations thereof, the 4-pyrone derivative being from a plant source different from said plant species.

Yet a further aspect provides for an add-on antimicrobial composition consisting of at least 4-pyrone derivative (e.g. maltol) and at least one plant extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material and any mixtures or combinations thereof, the 4-pyrone derivative being from a plant source different from said plant species.

In all of the aspects disclosed herein, the 4-pyrone derivative, in some embodiments, is obtained from a plant source different from said plant species.

The term add-on composition (or add-on formulation) is meant to refer to a composition that is added to another, already-prepared composition. For example, the add-on composition may be added to a variety of other products, e.g. shampoo, soap, cream, lotion, etc., to provide these products with a desired property. Such property may be antimicrobial activity, foaming, viscosity modification, improved absorbance, and the like. It is of note that the add-on formulation may be formulated separately from the composition of the product, and then added to the product. However, it is to be understood that each of the components of the add-on formulation may be individually added to the composition of the product at any desired addition sequence.

The composition of this disclosure may be added-to or formulated into various formulations which require preservation, disinfection or reduction in bacterial contaminant. In such formulations, the content of the composition may be between about 0.0001 and about 2 wt % of the formulation. In some embodiments, the content of the composition may be between about 0.0001 and about 1.5 wt %, between about 0.0001 and about 1 wt %, or even between about 0.0001 and about 0.8 wt % of the formulation. In other embodiments, the content of the composition may be between about 0.001 and about 2 wt %, between about 0.01 and about 2 wt %, or even between about 0.1 and about 2 wt % of the formulation. In some other embodiments, the content of the composition may be between about 0.001 and about 1.5 wt %, between about 0.01 and about 1 wt %, or even between about 0.1 and about 0.8 wt % of the formulation.

By another aspect, there is provided a composition having an antimicrobial activity, selected from:
(a) a composition comprising Styrax extract and maltol;
(b) a composition comprising Styrax extract, maltol, and Nigella sativa extract;
(c) a composition comprising Styrax extract, maltol, and Zingiber officinale extract;
(d) a composition comprising Styrax extract, maltol, and Cinnamomum cassia extract;
(e) a composition comprising Styrax extract, maltol, Cinnamomum cassia extract, and citric acid;
(f) a composition comprising Styrax extract, maltol, Paeonia lactiflora extract, and Terminalia ballerica extract;
(g) a composition comprising Styrax extract, maltol, Paeonia lactiflora extract, Terminalia ballerica extract and citric acid;
(h) a composition comprising maltol and Paeonia lactiflora extract;
(i) a composition comprising maltol and Terminalia ballerica extract;
(j) a composition comprising maltol and Magnolia officinalis extract;
(k) a composition comprising maltol and Cymbopogon citratus (lemongrass) extract;
(l) a composition comprising maltol and Salvia officinalis leaves extract;
(m) a composition comprising maltol and Nigella sativa extract;
(n) a composition comprising maltol and Pimenta dioica (allspice) extract;
(o) a composition comprising maltol and Cinnamomum cassia extract;
(p) a composition comprising maltol and Malpighia emarginata (acerola) extract;
(q) a composition comprising maltol and Origanum vulgare extract;
(r) a composition comprises maltol and Sapindus mukorossi extract;
(s) a composition comprising maltol and Camelia oleifera extract;
(t) a composition comprising maltol and Syzygium aromaticum (clove) extract; and
(u) a composition comprising maltol and Thymus vulgaris extract.

By some embodiments, the composition comprises at least 0.5 wt % Styrax extract and at least 0.05 wt % maltol.

By other embodiments, the composition comprises at least 0.12 wt % maltol and at least 0.0004 wt % Cinnamomum cassia extract.

The compositions of this disclosure may be prepared by any commonly used method for preparing a composition of materials. For example, the components of the compositions may be added as solids and mixed together, or one of the components may be added to the other in the form of a solution which may, if desired be evaporated or lyophilized after mixing for obtaining a homogeneous solution.

As will be further demonstrated below, the compositions of the disclosure exhibit antimicrobial properties which render the compositions suitable for a variety of applications in the fields of, e.g., cosmetics, therapeutics, foodstuffs and as material preservation.

The composition of this disclosure may thus be formulated into a variety of formulations, such as a cosmetic formulation, a therapeutic formulation, an antimicrobial formulation, a food additive formulation and a preservative formulation. Each of the aforementioned formulations may further comprise an excipient, diluents, or carrier suitable for the particular application, together with at least one additional additive as disclosed herein.

In another of its aspects, the present disclosure provides a cosmetic or cleansing formulation comprising compositions of the present disclosure as defined in the various embodiments hereinabove.

The cosmetic/cleansing formulations according to this disclosure can typically be formulated in a form adapted for topical application comprising a cosmetically or dermatologically acceptable medium, namely a medium which is suitable for application onto the skin of a subject (human or non-human). The medium may be in the form of aqueous or hydroalcoholic solution, an oil-in-water or water-in-oil emulsion, a microemulsion, aqueous or anhydrous gels, serum, a dispersion of vesicles, a patch, cream, spray, salve, ointment, lotion, gel, solution, suspension, or any other known cosmetically acceptable form. The formulation may alternatively be formulated for application to the human skin, hair, eyelashes, eyebrows, or nails.

In addition, the formulation may contain other standard additives such as an emollient, moisturizer, thickener, emulsifier, neutralizer, coloring agent, a fragrance, absorber or filter, preservative and/or gelling agent such as those described below, filler such as nylon, a sunscreen agent, electrolytes, proteins, antioxidants and chelating agents.

The formulation may also further comprise at least one active ingredient such as peptide active ingredients, vegetable extracts, anti-age agents, anti-wrinkle agents, soothing agents, radical scavengers, UV absorbing agents, agents stimulating the synthesis of dermal macromolecules or the energy metabolism, hydrating agents, anti-bacterial agents, anti-fungal agents, anti-inflammatory agents, anesthetic agents, agents modulating cutaneous differentiation, pigmentation or de-pigmentation, agents stimulating nail or hair growth, etc.

In some embodiments, each of the aforementioned additives/active ingredients is generally present in an amount of between about 0.1 and 30 wt % of the total weight of the formulation.

Suitable emollients for use in a cosmetic/cleansing formulation according to this disclosure include, for example, optionally hydroxy-substituted C8-C50 unsaturated fatty acids and esters thereof, C1-C24 esters of C8-C30 saturated fatty acids such as isopropyl myristate, cetyl palmitate and octyldodecylmyristate (Wickenol 142), beeswax, saturated and unsaturated fatty alcohols such as behenyl alcohol and cetyl alcohol, hydrocarbons such as mineral oils, petrolatum, squalane, fatty sorbitan esters, lanolin and lanolin derivatives, such as lanolin alcohol ethoxylated, hydroxylated and acetylated lanolins, cholesterol and derivatives thereof, animal and vegetable triglycerides such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppy seed oil, pine oil, castor oil, soybean oil, avocado oil, safflower oil, coconut oil, hazelnut oil, olive oil, grape seed oil, and sunflower seed oil and C1-C24 esters of dimer and trimer acids such as diisopropyl dimerate, diisostearylmalate, diisostearyldimerate and triisostearyltrimerate.

In some embodiments, the emollients used in a formulation can include isocetyl alcohol, octyl palmitate, isostearyl neopentanoate and isocetyl stearyl stearate, natural or synthetic oils selected from mineral, vegetable, and animal oils, fats and waxes, fatty acid esters, fatty alcohols, alkylene glycol and polyalkylene glycol ethers and esters, fatty acids and mixtures thereof.

The emollients may be used independently or in mixtures and may be present in the composition in an amount from about 1 to about 98% by weight, and in some embodiments are present in an amount from about 5% to about 15% by weight of the total formulation.

Suitable emulsifiers for use in a cosmetic/cleansing formulation according to the present disclosure include glyceryl stearate and laureth 23, PEG 20 stearate, and mink-amidopropyl dimethyl 2-hydroxyethylammonium chloride.

Typical moisturizers are glycerin, petrolatum and maleated vegetable oil.

The formulation may also contain a hydrophilic gelling agent, which may, by some embodiments, be selected from water-soluble or colloidal water-soluble polymers, such as cellulose ethers (e.g. hydroxyethyl cellulose, methyl cellulose, hydroxypropylmethyl cellulose), polyvinylalcohol, polyquaternium-10, guar gum, hydroxypropyl guar gum, xanthan gum, Aloe vera gel, amla, carrageenan, oat flour, starch (from corn rice or other plants), gelatin (porcine skin), ghatty gum, gum Arabic, inulin (from chicory), Konjac gum, locust bean gum, marshmallow root, pectin, quinoa extract, red alga, solagum and tragacanth gum (TG).

In further embodiments, the hydrophilic gelling agents are selected amongst acrylic acid/ethyl acrylate copolymers and the carboxyvinyl polymers (Carbopol). These resins consist essentially of a colloidal water-soluble polyalkenyl polyether crosslinked polymer of acrylic acid crosslinked with from 0.75% to 2.00% of a crosslinking agent such as polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1 of polyallyl ether of sucrose having an average of about 5.8 allyl groups for each sucrose molecule. Also suitable for use herein are hydrophobically-modified crosslinked polymers of acrylic acid having amphipathic properties available under the Trade Name Carbopol 1382, Carbopol 1342 and Pemulen TR-1. A combination of the polyalkenyl polyether cross-linked acrylic acid polymer and the hydrophobically modified crosslinked acrylic acid polymer is also suitable for use herein.

Other suitable gelling agents suitable for use herein are oleogels such as trihydroxystearin and aluminum magnesium hydroxy stearate.

In some embodiments, the gelling agent is present in the cosmetic/cleansing formulation in an amount from about 0.01% to about 10% of the total weight of the formulation. In some embodiments, the formulation comprises a hydrophilic gelling agent in an amount between about 0.02% to about 2%. In other embodiments, the amount of the gelling agent is from about 0.02% to about 0.5%.

The cosmetic/cleansing formulation may also comprise a thickener, such as crosslinked maleic anhydride-alkyl methylvinylethers, and copolymers, commercially available as Stabilizes QM (International Specialty Products (ISP)), Carbomer, natural gums, highly crosslinked polymethacrylate copolymer, etc.

Neutralizing agents suitable for use in a cosmetic/cleansing formulation include neutralizing acidic group containing hydrophilic gelling agents, as listed herein, sodium hydroxide, potassium hydroxide, ammonium hydroxide, monoethanolamine, diethanolamine and triethanolamine and aminomethyl propanol.

In some embodiments, the cosmetic/cleansing formulation comprises one or more ultraviolet absorbing agents. Ultraviolet absorbing agents, often described as sun screening agents, may be present in a concentration between about 1% and about 25% by weight, based on the total weight of composition. According to some embodiments, the UV absorbing agent constitutes between about 2% and 15% by weight. According to other embodiments, the UV absorbing agent constitutes between about 4% and about 10% by weight. Non-limiting examples of ultraviolet absorbing agents include benzophenone-3, benzophenone-4, octyl dimethyl PABA (Padimate 0), octyl methoxy cinnamate, octyl salicylate, octocrylene, p-methylbenzylidene camphor, butyl methoxy dibenzoyl methane (Parsol 1789), titanium dioxide, zinc oxide and mixtures thereof.

The antimicrobial compositions of this disclosure are effective in reducing or eliminating a microorganism population or a biofilm of such microorganisms. As demonstrated herein, the compositions provide antimicrobial activity against a wide spectrum of microorganisms and specifically against a broad spectrum of bacteria. The term microorganism relates herein to a single cell (unicellular), cell clusters, or no cell (acellular) organism such as bacteria, fungi, yeast, mold, archaea, protists, viruses and algae.

In some embodiments, the microorganism is a bacteria, being selected from Acinetobacter baumannii, Anaerococcus sp, Anaerococcus prevotii, Bordetella pertussis, Borrelia burgdorferi, Brucella abortus, Brucella canis, Brucella melitensis, Brucella suis, Burkholderia cepacia, Campylobacter jejuni, Chlamydia pneumonia, Chlamydia psittaci, Chlamydia trachomatis, Clostridium botulinum, Clostridium difficile, Clostridium perfringens, Clostridium tetani, Corynebacterium diphtheria, Cutibacterium acnes, Enterobacter cloacae, Enterobacter cloacae, Enterococcus faecalis, Enterococcus faecium, Escherichia coli (E. coli), Enterotoxigenic Escherichia coli (ETEC), Enteropathogenic E. coli, Francisella tularensis, Gardnerella vaginalis, Haemophilus influenza, Halomonas elongate, Helicobacter pylori, Klebsiella oxytoca, Lactobacillus acidophilus, Legionella pneumophila, Leptospira interrogans, Listeria monocytogenes, Mycobacterium leprae, Mycobacterium tuberculosis, Mycoplasma pneumonia, Neisseria gonorrhoeae, Neisseria meningitidis, Pseudomonas aeruginosa, Rickettsia rickettsii, Salmonella typhi, Salmonella typhimurium, Shigella sonnei, Staphylococcus capitis, Staphylococcus epidermidis, Staphylococcus saprophyticus, Streptococcus agalactiae, Streptococcus mutans Streptococcus pneumonia, Streptococcus pyogenes, Treponema pallidum, Vibrio cholera, Vibrio harveyi and Yersinia pestis.

In some embodiments, the microorganism is a fungus, selected from Absidia corymbifera, Ajellomyces capsulatus, Ajellomyces dermatitidis, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger (Aspergillus brasiliensis), Aspergillus terreus, Blastomyces dermatitidis, Candida albicans, Candida albicans var. stellatoidea, Candida dublinensis, Candida glabrata, Candida guilliermondii, Candida krusei, Candida parapsilosis, Candida pelliculosa, Candida tropicalis, Cladophialophora carrionii, Coccidioides immitis, Cryptococcus neoformans, Cunninghamella sp., Epidermophyton floccosum, Exophiala dermatitidis, Filobasidiella neoformans, Fonsecaea pedrosoi, Geotrichum candidum, Histoplasma capsulatum, Hortaea werneckii, Issatschenkia orientalis, Madurella grisae, Malassezia furfur, Malassezia furfur complex, Malassezia globosa, Malassezia obtuse, Malassezia pachydermatis, Malassezia restricta, Malassezia slooffiae, Malassezia sympodialis, Microsporum canis, Microsporum fulvum, Microsporum gypseum, Microsporum gypseum complex, Microsporum gypseum, Mucor circinelloides, Nectria haematococca, Paecilomyces variotii, Paracoccidioides brasiliensis, Penicillium marneffei, Phialophora verrucosa, Pichia anomala, Pichia guilliermondii, Pneumocystis jirovecii, Pseudallescheria boydii, Rhizopus oryzae, Rodotorula rubra, Saccharomyces cerevisiae, Scedosporium apiospermum, Schizophyllum commune, Sporothrix schenckii, Stachybotrys chartarum, Trichophyton mentagrophytes, Trichophyton mentagrophytes complex, Trichophyton mentagrophytes, Trichophyton mentagrophytes, Trichophyton rubrum, Trichophyton tonsurans, Trichophyton verrucosum, Trichophyton violaceum, Trichosporon asahii, Trichosporon cutaneum, Trichosporon cutaneum complex, Trichosporon inkin, Trichosporon mucoides and Wickerhamomyces anomalus.

In some embodiments, the microorganism is yeast, being selected from Candida albicans, Candida albicans var. stellatoidea, Candida dublinensis, Candida glabrata, Candida guilliermondii, Candida krusei, Candida parapsilosis, Candida pelliculosa, Candida tropicalis, Cryptococcus neoformans, Filobasidiella neoformans, Geotrichum candidum, Issatschenkia orientalis, Malassezia furfur, Malassezia pachydermatis, Pichia anomala, Pichia guilliermondii, Pityrosporum ovale, Pneumocystis jirovecii, Rodotorula rubra, Trichosporon asahii, Trichosporon cutaneum, Trichosporon inkin and Trichosporon mucoides.

In some embodiments, the microorganism is mold, being selected from Absidia corymbifera, Arthroderma benhamiae, Arthroderma fulvum, Arthroderma gypseum, Arthroderma incurvatum, Arthroderma otae, Arthroderma vanbreuseghemii, Aspergillus flavus, Aspergillus fumigates, Aspergillus niger, Cladophialophora carrionii, Coccidioides immitis, Epidermophyton floccosum, Exophiala dermatitidis, Fonsecaea pedrosoi, Hortaea werneckii, Madurella grisae, Microsporum canis, Microsporum fulvum, Microsporum gypseum, Microsporum gypseum, Microsporum gypseum, Mucor circinelloides, Nectria haematococca, Paecilomyces variotii, Paracoccidioides brasiliensis, Penicillium marneffei, Pseudallescheria boydii, Rhizopus oryzae, Scedosporium apiospermum, Schizophyllum commune, Sporothrix schenckii, Stachybotrys chartarum, Trichophyton mentagrophytes complex, Trichophyton mentagrophytes, Trichophyton mentagrophytes, Trichophyton rubrum, Trichophyton tonsurans, Trichophyton verrucosum and Trichophyton violaceum.

According to some embodiments of this disclosure, the antimicrobial compositions disclosed herein are effective against bacteria such as Escherichia coli (E. Coli), Salmonella, Staphylococcus, Saccharomyces, Staphylococcus aureus, Pseudomonas aeruginosa, Candida albicans, A. niger, L. acidophilus, Aspergillus terreus, Klebsiella oxytoca, Penicillium marneffei, Acinetobacter baumannii, Pityrosporum ovale, Staphylococcus capitis, Wickerhamomyces anomalus, Staphylococcus saprophyticus, Anaerococcus prevotii, Staphylococcus epidermidis, Streptococcus mutans, Cutibacterium acnes, Halomonas elongate, and/or Gardnerella vaginalis.

According to some embodiments of this disclosure, the antimicrobial compositions disclosed herein are effective against bacteria such as Escherichia coli (E. Coli), Salmonella, Staphylococcus, Saccharomyces, Staphylococcus aureus, Pseudomonas aeruginosa, Candida albicans, A. niger.

By some embodiments, the compositions of this disclosure exhibit delayed antimicrobial activity. In other words, the compositions are designed to eliminate undesired microorganisms in the long term (for example, after a few days from exposure), however have no effect, or have a significantly reduced effect, when exposing the microorganisms to the composition for short periods of time. The compositions do not have significant antimicrobial effect in short-term exposure, making the compositions friendly to the skin microbiota for a period of time suitable for the application of the composition onto the skin, however showing an antimicrobial effect after longer exposure, for example in storing conditions of the cosmetic product into which the compositions are combined (e.g. creams, lotions, gels, etc.). Such delayed antimicrobial effect enables providing a composition which can be used to preserve cosmetic formulations for a prolonged period of time against the growth of undesired microbiological contaminants (such as Escherichia coli (E. Coli), Staphylococcus aureus, Pseudomonas aeruginosa, Candida albicans, A. niger) when the microorganisms are continuously exposed to the composition, while maintaining the desired and beneficial microbiota of the skin (e.g. S. capitis, S. epidermis or S. hominis) when the cosmetic formulation is applied thereto.

Therefore, by another aspect of this disclosure, there is provided a composition having a delayed antimicrobial activity, the composition comprising at least 4-pyrone derivative (e.g. maltol) and at least one plant extract from at least one plant species of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and any mixtures or combinations thereof.

By some embodiments, the composition shows antimicrobial activity observable from at least 5 hours of continuous exposure of the microorganisms to the composition. By other embodiments, the composition shows antimicrobial activity observable from at least 8 hours, 12 hours, 24 hours, 2-days, 3-days, 4-days or even 5-days of continuous exposure of the microorganisms to the composition.

In another of its aspects, the present disclosure provides an oral care formulation comprising compositions of the present disclosure as defined in the various embodiments hereinabove. The oral care formulation may be in any suitable form, such as toothpaste, mouthwash, lozenges, chewing gum, dissolvable patches, dissolvable strips, etc. The oral care formulation may be effective in reducing, delaying and even preventing formation of a biofilm onto one or more surfaces of the oral cavity, e.g. teeth, gums, tung, cheeks, etc. The oral care formulation due its antimicrobial activity, may also reduce or prevent proliferation of caries-causing bacteria (such as Streptococcus mutans, Streptococcus sobrinus, and various lactobacilli), which secrete various acidic decomposition products that induce tooth decay and enamel damage. [In a further aspect, the disclosure provides a therapeutic formulation (pharmaceutical composition) comprising the compositions of the present disclosure as described herein.

The pharmaceutical formulation disclosed herein may be effective in the treatment and/or prevention of a variety of diseases and disorders. As demonstrated hereinbelow, the formulations provide instant and persistent antimicrobial activity against a wide spectrum of microorganisms, as defined herein. In some embodiments, the disease or disorder to be treated is associated with bacterial infection, fungal infection or viral infection.

Non-limiting examples of disease or disorder associated with a bacterial infection include lyme disease, brucellosis, acute enteritis, psittacosis, nongonococcal urethritis (NGU), trachoma, inclusion conjunctivitis of the newborn (ICN), lymphogranuloma venereum (LGV), botulism, pseudomembranous colitis, gas gangrene, acute food poisoning, anaerobic cellulitis, tetanus, diphtheria, nosocomial infections, urinary tract infections (UTI), diarrhea, meningitis, meningitis in infants, hemorrhagic colitis, hemolytic-uremic syndrome, tularemia, upper respiratory tract infections, pneumonia, mycoplasma pneumonia, secondary pneumonia, bronchitis, peptic ulcer, legionnaire's disease, gastric B-cell lymphoma, pontiac fever, leptospirosis, listeriosis, leprosy (Hansens disease), tuberculosis, gonorrhea, ophthalmia neonatorum, meningococcal disease, Waterhouse-Friderichsen, localized infection (of eye, ear, skin, urinary, respiratory), dental caries, gastrointestinal tract infection, central nervous system infection, systemic infection with bacteremia, bone and joint infections, endocarditis, typhoid fever type salmonellosis, dysentery, colitis, salmonellosis with gastroenteritis and enterocolitis, bacillary dysentery/shigellosis, Streptococcal pharyngitis, Scarlet fever, rheumatic fever, impetigo and erysipelas, puerperal fever, necrotizing fasciitis, syphilis, congenital syphilis and cholera.

In some embodiments, the bacterial disease or disorder is associated with Staphylococcus or Escherichia coli (E. coli) or Salmonella infections; the disease or disorder being selected from:
Staphylococcus: coagulase-positive staphylococcal infections such as localized skin infections, diffuse skin infection (impetigo), deep and localized infections, acute infective endocarditis, septicemia, necrotizing pneumonia, toxinoses, toxic shock syndrome, staphylococcal food poisoning, infections of implanted prostheses e.g. heart valves and catheters and cystitis in women;
E. coli: urinary tract infections (UTI), diarrhea, meningitis in infants, traveler's diarrhea, hemorrhagic colitis and hemolytic-uremic syndrome;
Salmonella: typhoid fever type salmonellosis, dysentery, colitis, salmonellosis, e.g. with gastroenteritis and enterocolitis.

In some embodiments, the pharmaceutical compositions of this disclosure are used in the treatment or prevention of a disease or disorder associated with a fungal infection. In some embodiments, the pathogen is yeast. In some other embodiments, the pathogen is mold.

The pharmaceutical composition may be adapted for administration by a variety of routes including topical, oral, dental, rectal, vaginal, transdermal, subcutaneous, intravenous, intramuscular, eye drops and intranasal. Such pharmaceutical composition is prepared in a manner well known in the pharmaceutical art. In making the pharmaceutical composition, the aforementioned components are usually mixed with an excipient, diluted by an excipient or enclosed within such a carrier which can be manipulated to the desired form. Based on the particular mode of administration, the pharmaceutical composition may be formulated into tablets, pills, capsules, sachets, granules, powders, chewing gum, suspensions, emulsions and solutions.

The pharmaceutically acceptable carriers, for example, vehicles, adjuvants, excipients, or diluents, are well-known to those who are skilled in the art and are readily available to the public. It is preferred that the pharmaceutically acceptable carrier be one which is chemically inert to the active formulation and each of its components and one which has no detrimental side effects or toxicity under the conditions of use.

The choice of carrier will be determined in part by the particular formulation, as well as by the particular method used to administer the composition. Accordingly, there is a wide variety of suitable formulations of the pharmaceutical composition of this disclosure.

Formulations suitable for oral administration can consist of (a) liquid solutions, such as an effective amount of the compound, or composition comprising same, dissolved in diluents, such as water, saline, or juice (e.g. orange juice); (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active formulation in an inert base, such as gelatin and glycerin, or sucrose and acacia, emulsions, gels, and the like containing, in addition to the active formulation, such carriers as are known in the art.

The formulations of the present disclosure, alone or in combination with other suitable components, e.g., active or non-active additives/ingredients can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The formulation can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical additives.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The active formulation is effective over a wide dosage range and may generally be administered in a pharmaceutically effective amount. It should be understood, however, that the amount of the formulation or each component thereof to be administered, will be determined by a physician, in the light of the relevant circumstances, including the condition to be treated, the chosen route of administration, the actual formulation, the age, weight, and response of the individual patient, the severity of the patient's symptoms, and the like.

In another aspect of the disclosure, there is provided the use of a formulation of the disclosure as herein defined, for the preparation of a pharmaceutical composition for treating or preventing a disease or disorder in a mammal (human or non-human).

In another aspect of this disclosure, there is provided the use of a topical formulation comprising the compositions defined herein, for the preparation of a pharmaceutical composition for treating or preventing a disease or disorder in a mammal (human or non-human).

In some embodiments, the disease or disorder is associated with a bacteria, virus, fungus, yeast or mold.

As used herein, the term treatment or any lingual variation thereof, refers to the administering of a therapeutic amount of the composition which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above. The effective amount for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

In yet another aspect, the present disclosure provides a preservative formulation comprising the compositions of the disclosure as described herein.

The compositions of this disclosure may, by some embodiments, be formulated as a preservative formulation, an antimicrobial formulation, a pharmaceutical composition, a disinfectant formulation, and oral care formulation and a cosmetic formulation.

The preservative formulation may be used to reduce, inhibit or completely eliminate pathogen population in a variety of consumer products, such as personal care products, industrial products, food products, therapeutics, and others. As demonstrated herein, the formulation may be used to replace currently available chemicals which are used as preservatives, some of which known as toxic to humans and animals, or at reduce their concentration in such products for human or animal use. The preservative formulation may be added to any such product, such as cosmetics and toiletries in aqueous or hydroalcoholic solution, oil-in-water or water-in-oil emulsion, aqueous or anhydrous gels, cream, ointment, lotion, gel, solution and suspension; therapeutics and over-the-counter pharmaceutical products, water-based paints, cutting oils, latex solutions, food products such as beverages, frozen foods, candy and canned products.

In some embodiments, the formulation is an antimicrobial preservative, attesting to the ability of the formulations to suppress microbial growth, reduce microbial infestation, treat products or surfaces to improve product resistance to microbial infestation, reduce biofilm, remove biofilm, prevent conversion of bacteria to biofilm, prevent or inhibit microbial infection, prevent spoilage, retard or minimize or prevent quorum sensing, and retard microbial reproduction. Typically, the preservative formulation according to this disclosure comprises at least one 4-pyrone derivative and plant extract(s) at a concentration which suffices to prevent spoilage or growth of microorganisms, thereby extending the shelf- or useful-life of the product.

The formulations of this disclosure may also be employed as a disinfectant or bactericidal agent. The formulations may be applied onto a surface to be disinfected, including human or animal skin, by various means including by washing, spraying, wiping, etc.

As used herein, the term about is meant to encompass deviation of +10% from the specifically mentioned value of a parameter, such as temperature, pressure, concentration, etc.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

For the avoidance of doubt, all concentrations of ingredients in this disclosure are provided as weight-per-weight (i.e. weight percent or wt %), unless otherwise explicitly and/or specifically noted.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are pictures of agar plates containing body lotion samples taken after 3-4 hours of incubation with a mixture of representative microorganisms from skin microbiota. The upper panel of each agar plate was plated with mixture of representative microorganisms to provide a control to each of the experiments. The lower panel of each agar plate represent cream (preserved or unpreserved) which was inoculated with the same amount of microorganism's mixture: FIG. 1A bottom part of the plate is an unpreserved cream; FIG. 1B bottom part of the plate shows use of a commercial synthetic preservative; and FIG. 1C bottom part of the plate shows use of a composition according to Example 6.

### DETAILED DESCRIPTION OF EMBODIMENTS

### Example 1: Composition of Styrax Extract and Maltol in Lotion Formulation

A Preservatives Effectiveness Test (PET) was carried out for various combinations of Styrax extract (Sumatra benzoin) and maltol (obtained from sugarcane fermentation) for evaluating the synergistic antimicrobial effect therebetween. The antimicrobial efficiency was tested in accordance with current ISO 11930 requirements. The test consists of challenging a reference lotion with a prescribed inoculum of suitable microorganisms. The tested microorganisms used for the PET are detailed in Table 1, while the various combinations of Styrax extract and maltol are detailed in Table 2.

TABLE 1
Tested microorganisms

| | | Culture | Culture Growth | PET Growth |
|---|---|---|---|---|
| Microorganism | ATCC | Media | Conditions | Conditions |
| Staphylococcus aureus (SA) | 6538 | TSA | 18-24 hr, | 3-5 days, |
| Escherichia coli | 8739 | | 32.5 ± 2.50 C. | 32.5 ± 2.50 C. |
| (EC) | | | | |
| Pseudomonas aeruginosa | 9027 | | | |
| (PA) | | | | |
| Candida albicans | 10231 | SDA | 48-72 hr, | 5-7 days, |
| (CA) | | | 22.5 ± 2.50 C. | 22.5 ± 2.50 C. |
| Aspergillus brasiliensis | 16404 | | 7 days, | |
| (AB) | | | 22.5 ± 2.50 C. | |

TABLE 2
wt % of Styrax extract and maltol in lotion formulation

| Formula No. | Styrax ext. | Maltol |
|---|---|---|
| 1 | 5.5 | 2 |
| 2 | 5.5 | — |
| 3 | 5.5 | 0.2 |
| 4 | — | 2 |
| 5 | 0.55 | 2 |
| 6 | 2.75 | 1 |
| 7 | 2.75 | — |
| 8 | 2.75 | 0.2 |
| 9 | — | 1 |
| 10 | 0.55 | 1 |
| 11 | — | 0.2 |
| 12 | 0.55 | 0.2 |
| 13 | 0.55 | — |
| 14 | 0.55 | 0.04 |
| 15 | — | 0.04 |
| 16 | 0.11 | 0.2 |
| 17 | 0.11 | — |
| 18 | 0.11 | 0.04 |
| 19 | 0.55 | 0.02 |
| 20 | — | 0.02 |
| 21 | 0.055 | 0.2 |
| 22 | 0.055 | — |
| 23 | 0.055 | 0.02 |

| | |
|---|---|
| 24 | 1% Synthetic preservative (positive control) |
| 25 | Lotion without additives (negative control) |

Each sample was inoculated at time “0” and mixed with one of the microbial suspensions using a ratio-equivalent to 1% inoculum to product (30 μl inoculum into 3 g sample). The concentrations of test microorganisms added to the lotion were such that the concentration in the lotion immediately after inoculation was between 1×10⁶-1×10⁷ CFU/g for bacteria and 1×10⁵-1×10⁶ CFU/g for molds. Samples from the challenged test product were drawn at specific time intervals and analyzed for microbial counts.

Samples were stored at 20-25° C. Each sample was sampled in intervals of 7, 14 and 28 days. At Day 7, 0.1 g of the sample were directly seeded on TSA plates for bacteria and on SDA plates for mold. For A. brasiliensis, a dilution of 1:10 was prepared (0.1 g of the inoculated lotion in 900 μL NaCl-peptone saline) and 0.1 μL of the 1:10 dilution was seeded on SDA plate. At Days 14 and 28, 0.1 g of the samples were directly seeded on TSA plates for bacteria and on SDA plates for molds.

The number of viable microorganisms/g in the mixture at each test interval was determined by the Spreading on agar Count Method. The TSA plates were incubated at 30-35° C. for 3-5 days unless a reliable count was obtained in a shorter time. The SDA plates were incubated at 20-25° C. for 5-7 days or less if reliable count was obtained prompter.

The counts obtained in all countable dilutions were used to calculate the mean value of CFU, per gram product/challenge-microorganism/time interval. Using the calculated concentration of microorganisms in the initial inoculum per gram present as a baseline, the change in log 10 values of the concentration of the CFU/g was calculated for each time interval and the changes were expressed in terms of log reduction.

$$Log Reduction = \frac{Log ( Initial count of each microorganism )}{Log ( countable microorganisms at each time interval )}$$

Count results are provided in Tables 3-1 to 3-4.

TABLE 3-1
Total count CFU/g, initial inoculum (Day 0)

| | A. | C. | P. | | |
|---|---|---|---|---|---|
| Formula | brasilliensis | albicans | aeruginosa | E. coli | S. aureus |
| 1 | 4.50E+05 | 1.50E+05 | 4.00E+06 | 4.00E+06 | 1.40E+07 |
| 2 | 4.50E+05 | 1.50E+05 | 4.00E+06 | 4.00E+06 | 1.40E+07 |
| 3 | 4.50E+05 | 1.50E+05 | 4.00E+06 | 4.00E+06 | 1.40E+07 |
| 4 | 4.50E+05 | 1.50E+05 | 4.00E+06 | 4.00E+06 | 1.40E+07 |
| 5 | 4.50E+05 | 1.50E+05 | 4.00E+06 | 4.00E+06 | 1.40E+07 |
| 6 | 4.50E+05 | 1.50E+05 | 4.00E+06 | 4.00E+06 | 1.40E+07 |
| 7 | 4.50E+05 | 1.50E+05 | 4.00E+06 | 4.00E+06 | 1.40E+07 |
| 8 | 4.50E+05 | 1.50E+05 | 4.00E+06 | 4.00E+06 | 1.40E+07 |
| 9 | 4.50E+05 | 1.50E+05 | 4.00E+06 | 4.00E+06 | 1.40E+07 |
| 10 | 4.50E+05 | 1.50E+05 | 4.00E+06 | 4.00E+06 | 1.40E+07 |
| 11 | 4.50E+05 | 1.50E+05 | 4.00E+06 | 4.00E+06 | 1.40E+07 |
| 12 | 4.50E+05 | 1.50E+05 | 4.00E+06 | 4.00E+06 | 1.40E+07 |
| 13 | 4.50E+05 | 1.50E+05 | 4.00E+06 | 4.00E+06 | 1.40E+07 |
| 14 | 4.50E+05 | 1.50E+05 | 4.00E+06 | 4.00E+06 | 1.40E+07 |
| 15 | 4.50E+05 | 1.50E+05 | 4.00E+06 | 4.00E+06 | 1.40E+07 |
| 16 | 4.50E+05 | 1.50E+05 | 4.00E+06 | 4.00E+06 | 1.40E+07 |
| 17 | 4.50E+05 | 1.50E+05 | 4.00E+06 | 4.00E+06 | 1.40E+07 |
| 18 | 4.50E+05 | 1.50E+05 | 4.00E+06 | 4.00E+06 | 1.40E+07 |
| 19 | 4.50E+05 | 1.50E+05 | 4.00E+06 | 4.00E+06 | 1.40E+07 |
| 20 | 4.50E+05 | 1.50E+05 | 4.00E+06 | 4.00E+06 | 1.40E+07 |
| 21 | 4.50E+05 | 1.50E+05 | 4.00E+06 | 4.00E+06 | 1.40E+07 |
| 22 | 4.50E+05 | 1.50E+05 | 4.00E+06 | 4.00E+06 | 1.40E+07 |
| 23 | 4.50E+05 | 1.50E+05 | 4.00E+06 | 4.00E+06 | 1.40E+07 |
| 24 | 4.50E+05 | 1.50E+05 | 4.00E+06 | 4.00E+06 | 1.40E+07 |
| 25 | 4.50E+05 | 1.50E+05 | 4.00E+06 | 4.00E+06 | 1.40E+07 |

TABLE 3-2
Total count CFU/g, Day 7

| | A. | C. | P. | | |
|---|---|---|---|---|---|
| Formula | brasilliensis | albicans | aeruginosa | E. coli | S. aureus |
| 1 | 0.00E+00 | 0.00E+00 | 0.00E+00 | 0.00E+00 | 0.00E+00 |
| 2 | 7.00E+02 | 1.50E+05 | 2.80E+03 | 0.00E+00 | 0.00E+00 |
| 3 | 0.00E+00 | 0.00E+00 | 0.00E+00 | 0.00E+00 | 0.00E+00 |
| 4 | 0.00E+00 | 0.00E+00 | 0.00E+00 | 0.00E+00 | 0.00E+00 |
| 5 | 0.00E+00 | 0.00E+00 | 0.00E+00 | 0.00E+00 | 0.00E+00 |
| 6 | 0.00E+00 | 0.00E+00 | 0.00E+00 | 0.00E+00 | 0.00E+00 |
| 7 | 2.20E+03 | 1.50E+05 | 4.00E+06 | 6.50E+02 | 0.00E+00 |
| 8 | 3.00E+01 | 0.00E+00 | 0.00E+00 | 0.00E+00 | 0.00E+00 |
| 9 | 0.00E+00 | 0.00E+00 | 0.00E+00 | 0.00E+00 | 0.00E+00 |
| 10 | 0.00E+00 | 0.00E+00 | 0.00E+00 | 0.00E+00 | 0.00E+00 |
| 11 | 3.24E+04 | 1.50E+05 | 0.00E+00 | 3.20E+03 | 1.40E+07 |
| 12 | 0.00E+00 | 4.00E+03 | 0.00E+00 | 0.00E+00 | 0.00E+00 |
| 13 | 8.00E+04 | 1.50E+05 | 4.00E+06 | 5.00E+03 | 0.00E+00 |
| 14 | 1.20E+03 | 1.50E+05 | 8.00E+01 | 4.00E+06 | 0.00E+00 |
| 15 | 4.50E+05 | 1.50E+05 | 4.00E+06 | 4.00E+06 | 1.40E+07 |
| 16 | 3.50E+03 | 4.80E+03 | 0.00E+00 | 0.00E+00 | 0.00E+00 |
| 17 | 2.40E+04 | 1.50E+05 | 4.00E+06 | 4.00E+06 | 0.00E+00 |
| 18 | 1.30E+03 | 1.50E+05 | 4.00E+06 | 4.00E+06 | 0.00E+00 |
| 19 | 3.50E+03 | 1.50E+05 | 4.00E+06 | 4.00E+06 | 0.00E+00 |
| 20 | 4.50E+05 | 1.50E+05 | 4.00E+06 | 4.00E+06 | 1.40E+07 |
| 21 | 5.00E+03 | 4.00E+04 | 0.00E+00 | 0.00E+00 | 0.00E+00 |
| 22 | 5.20E+04 | 1.50E+05 | 4.00E+06 | 4.00E+06 | 0.00E+00 |
| 23 | 1.20E+04 | 1.50E+05 | 4.00E+06 | 4.00E+06 | 0.00E+00 |
| 24 | 0.00E+00 | 0.00E+00 | 0.00E+00 | 0.00E+00 | 0.00E+00 |
| 25 | 4.50E+05 | 1.50E+05 | 4.00E+06 | 4.00E+06 | 1.40E+07 |

TABLE 3-3
Total count CFU/g, Day 14

| | A. | C. | P. | | |
|---|---|---|---|---|---|
| Formula | brasilliensis | albicans | aeruginosa | E. coli | S. aureus |
| 1 | 0.00E+00 | 0.00E+00 | 0.00E+00 | 0.00E+00 | 0.00E+00 |
| 2 | 3.20E+02 | 1.50E+05 | 4.00E+06 | 0.00E+00 | 0.00E+00 |
| 3 | 0.00E+00 | 0.00E+00 | 0.00E+00 | 0.00E+00 | 0.00E+00 |
| 4 | 0.00E+00 | 0.00E+00 | 0.00E+00 | 0.00E+00 | 0.00E+00 |
| 5 | 0.00E+00 | 0.00E+00 | 0.00E+00 | 0.00E+00 | 0.00E+00 |
| 6 | 0.00E+00 | 0.00E+00 | 0.00E+00 | 0.00E+00 | 0.00E+00 |
| 7 | 5.10E+02 | 1.50E+05 | 4.00E+06 | 4.00E+06 | 0.00E+00 |
| 8 | 2.00E+01 | 0.00E+00 | 0.00E+00 | 0.00E+00 | 0.00E+00 |
| 9 | 0.00E+00 | 0.00E+00 | 0.00E+00 | 0.00E+00 | 0.00E+00 |
| 10 | 0.00E+00 | 0.00E+00 | 0.00E+00 | 0.00E+00 | 0.00E+00 |
| 11 | 3.50E+03 | 0.00E+00 | 0.00E+00 | 0.00E+00 | 6.70E+06 |
| 12 | 3.00E+01 | 0.00E+00 | 0.00E+00 | 0.00E+00 | 0.00E+00 |
| 13 | 1.30E+04 | 1.50E+05 | 4.00E+06 | 4.50E+02 | 0.00E+00 |
| 14 | 3.00E+01 | 1.50E+05 | 4.00E+06 | 1.41E+03 | 0.00E+00 |
| 15 | 1.00E+05 | 1.50E+05 | 4.00E+06 | 2.00E+03 | 6.70E+06 |
| 16 | 1.10E+02 | 0.00E+00 | 0.00E+00 | 0.00E+00 | 0.00E+00 |
| 17 | 1.12E+04 | 1.50E+05 | 4.00E+06 | 4.00E+06 | 0.00E+00 |
| 18 | 4.00E+01 | 1.50E+05 | 4.00E+06 | 3.10E+02 | 0.00E+00 |
| 19 | 7.10E+02 | 1.50E+05 | 4.00E+06 | 5.50E+03 | 0.00E+00 |
| 20 | 1.30E+05 | 1.50E+05 | 4.00E+06 | 4.00E+06 | 6.70E+06 |
| 21 | 5.00E+02 | 0.00E+00 | 0.00E+00 | 0.00E+00 | 0.00E+00 |
| 22 | 2.10E+04 | 1.50E+05 | 4.00E+06 | 4.00E+06 | 0.00E+00 |
| 23 | 2.50E+03 | 1.50E+05 | 4.00E+06 | 3.00E+01 | 0.00E+00 |
| 24 | 0.00E+00 | 0.00E+00 | 0.00E+00 | 0.00E+00 | 0.00E+00 |
| 25 | 4.50E+05 | 1.50E+05 | 4.00E+06 | 4.00E+06 | 6.70E+06 |

TABLE 3-4
Total count CFU/g, Day 28

| | A. | C. | P. | | |
|---|---|---|---|---|---|
| Formula | brasilliensis | albicans | aeruginosa | E. coli | S. aureus |
| 1 | 0.00E+00 | 0.00E+00 | 0.00E+00 | 0.00E+00 | 0.00E+00 |
| 2 | 0.00E+00 | 3.60E+03 | 4.00E+06 | 0.00E+00 | 0.00E+00 |
| 3 | 0.00E+00 | 0.00E+00 | 0.00E+00 | 0.00E+00 | 0.00E+00 |
| 4 | 0.00E+00 | 0.00E+00 | 0.00E+00 | 0.00E+00 | 0.00E+00 |
| 5 | 0.00E+00 | 0.00E+00 | 0.00E+00 | 0.00E+00 | 0.00E+00 |
| 6 | 0.00E+00 | 0.00E+00 | 0.00E+00 | 0.00E+00 | 0.00E+00 |
| 7 | 0.00E+00 | 3.20E+03 | 4.00E+06 | 1.70E+02 | 0.00E+00 |
| 8 | 0.00E+00 | 0.00E+00 | 0.00E+00 | 0.00E+00 | 0.00E+00 |
| 9 | 0.00E+00 | 0.00E+00 | 0.00E+00 | 0.00E+00 | 0.00E+00 |
| 10 | 0.00E+00 | 0.00E+00 | 0.00E+00 | 0.00E+00 | 0.00E+00 |
| 11 | 2.30E+02 | 0.00E+00 | 0.00E+00 | 0.00E+00 | 0.00E+00 |
| 12 | 0.00E+00 | 0.00E+00 | 0.00E+00 | 0.00E+00 | 0.00E+00 |
| 13 | 5.00E+03 | 1.50E+05 | 4.00E+06 | 8.40E+03 | 0.00E+00 |
| 14 | 8.00E+01 | 1.50E+05 | 4.00E+06 | 0.00E+00 | 0.00E+00 |
| 15 | 4.50E+05 | 1.50E+05 | 4.00E+06 | 2.00E+02 | 5.80E+02 |
| 16 | 0.00E+00 | 0.00E+00 | 0.00E+00 | 0.00E+00 | 0.00E+00 |
| 17 | 5.00E+03 | 1.50E+05 | 4.00E+06 | 4.00E+06 | 0.00E+00 |
| 18 | 3.20E+03 | 1.50E+05 | 4.00E+06 | 1.00E+01 | 0.00E+00 |
| 19 | 7.00E+01 | 1.50E+05 | 4.00E+06 | 4.00E+06 | 0.00E+00 |
| 20 | 4.50E+05 | 1.50E+05 | 4.00E+06 | 1.05E+04 | 1.10E+02 |
| 21 | 2.00E+01 | 0.00E+00 | 0.00E+00 | 0.00E+00 | 0.00E+00 |
| 22 | 4.50E+05 | 1.50E+05 | 4.00E+06 | 4.00E+06 | 0.00E+00 |
| 23 | 2.00E+03 | 1.50E+05 | 4.00E+06 | 5.00E+01 | 0.00E+00 |
| 24 | 0.00E+00 | 0.00E+00 | 0.00E+00 | 0.00E+00 | 0.00E+00 |
| 25 | 4.50E+05 | 1.50E+05 | 4.00E+06 | 4.00E+06 | 6.70E+06 |

The summary of the results for the tested combinations, expressed in log reduction values, is provided in Table 4-1. Results (log reduction) of each of Styrax extract and Maltol are provided in Table 4-2 for comparison. In the Tables below, AB, CA, PA, EC and SA indicate A. brasilliensis, C. albicans, P. aeruginosa, E. coli and S. aureus, respectively.

TABLE 4-1
Summary of results for Styrax extract (benzoin) and maltol
combinations, log reduction from initial inoculation

| Formula | | | 7 Days | | | | | 14 Days | | | | | 28 Days | | | | |
|---|---|---|---|---|---|---|---|---|---|---|---|---|---|---|---|---|---|
| # | BZ (wt %) | ML (wt %) | AB | CA | PA | EC | SA | AB | CA | PA | EC | SA | AB | CA | PA | EC | SA |
| 1 | 5.5 | 2 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 2 | 5.5 | — | 2.8 | 0.0 | 3.2 | 6.6 | 7.1 | 3.1 | 0.0 | 0.0 | 6.6 | 6.8 | 5.7 | 1.6 | 0.0 | 6.6 | 6.8 |
| 3 | 5.5 | 0.2 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 4 | — | 2 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 5 | 0.55 | 2 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 6 | 2.75 | 1 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 7 | 2.75 | — | 2.3 | 0.0 | 0.0 | 3.8 | 7.1 | 2.9 | 0.0 | 0.0 | 0.0 | 6.8 | 5.7 | 1.7 | 0.0 | 4.4 | 6.8 |
| 8 | 2.75 | 0.2 | 4.2 | 5.2 | 6.6 | 6.6 | 7.1 | 4.4 | 5.2 | 6.6 | 6.6 | 6.8 | 5.7 | 5.2 | 6.6 | 6.6 | 6.8 |
| 9 | — | 1 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 10 | 0.55 | 1 | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 11 | — | 0.2 | 1.1 | 0.0 | 6.6 | 3.1 | 0.0 | 2.1 | 5.2 | 6.6 | 6.6 | 0.0 | 3.3 | 5.2 | 6.6 | 6.6 | 6.8 |
| 12 | 0.55 | 0.2 | 5.7 | 1.6 | 6.6 | 6.6 | 7.1 | 4.2 | 5.2 | 6.6 | 6.6 | 6.8 | 5.7 | 5.2 | 6.6 | 6.6 | 6.8 |
| 13 | 0.55 | — | 0.8 | 0.0 | 0.0 | 2.9 | 7.1 | 1.5 | 0.0 | 0.0 | 3.9 | 6.8 | 2.0 | 0.0 | 0.0 | 2.7 | 6.8 |
| 14 | 0.55 | 0.04 | 2.6 | 0.0 | 4.7 | 0.0 | 7.1 | 4.2 | 0.0 | 0.0 | 3.5 | 6.8 | 3.8 | 0.0 | 0.0 | 6.6 | 6.8 |
| 15 | — | 0.04 | 0.0 | 0.0 | 0.0 | 0.0 | 0.0 | 0.7 | 0.0 | 0.0 | 3.3 | 0.0 | 0.0 | 0.0 | 0.0 | 4.3 | 4.1 |
| 16 | 0.11 | 0.2 | 2.1 | 1.5 | 6.6 | 6.6 | 7.1 | 3.6 | 5.2 | 6.6 | 6.6 | 6.8 | 5.7 | 5.2 | 6.6 | 6.6 | 6.8 |
| 17 | 0.11 | — | 1.3 | 0.0 | 0.0 | 0.0 | 7.1 | 1.6 | 0.0 | 0.0 | 0.0 | 6.8 | 2.0 | 0.0 | 0.0 | 0.0 | 6.8 |
| 18 | 0.11 | 0.04 | 2.5 | 0.0 | 0.0 | 0.0 | 7.1 | 4.1 | 0.0 | 0.0 | 4.1 | 6.8 | 2.1 | 0.0 | 0.0 | 5.6 | 6.8 |
| 19 | 0.55 | 0.02 | 2.1 | 0.0 | 0.0 | 0.0 | 7.1 | 2.8 | 0.0 | 0.0 | 2.9 | 6.8 | 3.8 | 0.0 | 0.0 | 0.0 | 6.8 |
| 20 | — | 0.02 | 0.0 | 0.0 | 0.0 | 0.0 | 0.0 | 0.5 | 0.0 | 0.0 | 0.0 | 0.0 | 0.0 | 0.0 | 0.0 | 2.6 | 4.8 |
| 21 | 0.055 | 0.2 | 2.0 | 0.6 | 6.6 | 6.6 | 7.1 | 3.0 | 5.2 | 6.6 | 6.6 | 6.8 | 4.4 | 5.2 | 6.6 | 6.6 | 6.8 |
| 22 | 0.055 | — | 0.9 | 0.0 | 0.0 | 0.0 | 7.1 | 1.3 | 0.0 | 0.0 | 0.0 | 6.8 | 0.0 | 0.0 | 0.0 | 0.0 | 6.8 |
| 23 | 0.055 | 0.02 | 1.6 | 0.0 | 0.0 | 0.0 | 7.1 | 2.3 | 0.0 | 0.0 | 5.1 | 6.8 | 2.4 | 0.0 | 0.0 | 4.9 | 6.8 |

| | | | | | | | | | | | | | | | | |
|---|---|---|---|---|---|---|---|---|---|---|---|---|---|---|---|---|
| 24 | Synthetic | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| | Preservative 1% | | | | | | | | | | | | | | | |
| 25 | Lotion 100% | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |

TABLE 4-2
Summary of results for each of Styrax extract (benzoin)
and maltol, log reduction from initial inoculation

| | 7 Days | | | | | 14 Days | | | | | 28 Days | | | | |
|---|---|---|---|---|---|---|---|---|---|---|---|---|---|---|---|
| | AB | CA | PA | EC | SA | AB | CA | PA | EC | SA | AB | CA | PA | EC | SA |
| Benzoin | 2.8 | 0.0 | 0.0 | 6.6 | 7.1 | 3.1 | 0.0 | 0.0 | 6.6 | 6.8 | 5.7 | 1.6 | 0.0 | 6.6 | 6.8 |
| 5.5% | | | | | | | | | | | | | | | |
| Benzoin | 2.3 | 0.0 | 0.0 | 3.8 | 7.1 | 2.9 | 0.0 | 0.0 | 0.0* | 6.8 | 5.7 | 1.7 | 0.0 | 4.4 | 6.8 |
| 2.75% | | | | | | | | | | | | | | | |
| Benzoin | 0.8 | 0.0 | 0.0 | 2.9 | 7.1 | 1.5 | 0.0 | 0.0 | 3.9 | 6.8 | 2.0 | 0.0 | 0.0 | 2.7 | 6.8 |
| 0.55% | | | | | | | | | | | | | | | |
| Benzoin | 1.3 | 0.0 | 0.0 | 0.0 | 7.1 | 1.6 | 0.0 | 0.0 | 0.0 | 6.8 | 2.0 | 0.0 | 0.0 | 0.0 | 6.8 |
| 0.11% | | | | | | | | | | | | | | | |
| Benzoin | 0.9 | 0.0 | 0.0 | 0.0 | 7.1 | 1.3 | 0.0 | 0.0 | 0.0 | 6.8 | 0.0 | 0.0 | 0.0 | 0.0 | 6.8 |
| 0.055% | | | | | | | | | | | | | | | |
| Maltol | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 2% | | | | | | | | | | | | | | | |
| Maltol | 5.7 | 5.2 | 6.6 | 6.6 | 7.1 | 5.7 | 5.2 | 6.6 | 6.6 | 6.8 | 5.7 | 5.2 | 6.6 | 6.6 | 6.8 |
| 1% | | | | | | | | | | | | | | | |
| Maltol | 1.1 | 0.0 | 6.6 | 3.1 | 0.0 | 2.1 | 5.2 | 6.6 | 6.6 | 0.0 | 3.3 | 5.2 | 6.6 | 6.6 | 6.8 |
| 0.2% | | | | | | | | | | | | | | | |
| Maltol | 0.0 | 0.0 | 0.0 | 0.0 | 0.0 | 0.7 | 0.0 | 0.0 | 3.3 | 0.0 | 0.0 | 0.0 | 0.0 | 4.3 | 4.1 |
| 0.04% | | | | | | | | | | | | | | | |
| Maltol | 0.0 | 0.0 | 0.0 | 0.0 | 0.0 | 0.5 | 0.0 | 0.0 | 0.0 | 0.0 | 0.0 | 0.0 | 0.0 | 2.6 | 4.8 |
| 0.02% | | | | | | | | | | | | | | | |

As can be seen from Tables 3-1 to 4-2, when examining the effect of Styrax extract alone, activity was shown against S. aureus from the lowest concentration of 0.055% to the highest concentration of 5.5%, obtaining total kill in all concentrations tested. For E. coli, activity was shown at concentrations higher than 0.055%. No activity against P. aeruginosa and C. albicans was detected. Activity against A. brasiliensis was only obtained at high concentrations (5.5% and 2.75%) in all time intervals.

The effects obtained for Maltol alone are as follows. Activity against S. aureus was only obtained at high concentrations (5.5% and 2.75%) in all time intervals; activity against E. coli was obtained at concentrations higher than 0.04%, for all time intervals; activity against P. aeruginosa and A. brasiliensis was obtained at concentrations higher than 0.2%, for all time intervals; and activity against C. albicans was obtained at concentrations higher than 1%, for all time intervals.

Combination of Benzoin 5.5% and Maltol 0.2%: The combination yielded 5.7 log reductions (total kill) for A. brasiliensis at time 7 and 14 days, whereas standalone ingredients, each ingredient yielded 2.8 and 1.1 correspondingly. For C. albicans, no log reduction was obtained for Benzoin and Maltol as separate ingredients however, 5.2 log reductions were detected when tested combined.

Combination of Benzoin 2.75% and maltol 0.2%: The combination yielded 4.2 log reductions for A. brasiliensis at time 7 and 4.4 at time 14 days, whereas standalone ingredients, each ingredient yielded 2.3 and 1.1 correspondingly. For C. albicans, no log reduction was obtained for Benzoin and Maltol as separate ingredients however, 5.2 log reductions were detected when tested combined. For E. coli, 6.6 log reductions were observed for the combination, when only 3.8 and 3.1 log reductions were obtained as separate components.

Combination of Benzoin 0.55% and maltol 0.2%: The combination yielded 5.7 log reductions for A. brasiliensis at time 7, 28 and 4.2 at time 14 days, whereas standalone ingredients, each ingredient yielded up to 1.1 log reductions. For C. albicans, no log reduction was obtained for Benzoin and Maltol as separate ingredients however, 1.6 log reductions were detected when tested combined. For E. coli, 6.6 log reductions were observed for the combination, when only up to 3.1 log reductions were obtained as separate components.

Combination of Benzoin 0.55% and maltol 0.04%: The combination yielded 2.6-3.8 log reductions for A. brasiliensis, whereas standalone ingredients, each ingredient yielded up to 0.8 log reductions on day 7.

Combination of Benzoin 0.11% and maltol 0.2%: The combination yielded 2.1-5.7 log reductions for A. brasiliensis, whereas standalone ingredients, each ingredient yielded up to 1.3 log reductions on day 7. For C. albicans, no log reduction was obtained for Benzoin and Maltol as separate ingredients however, 1.5 log reductions at day 7 were detected when tested combined. For E. coli, 6.6 log reductions on day 7 were observed for the combination, when only up to 3.1 log reductions were obtained as separate components.

Combination of Benzoin 0.055% and maltol 0.2%: The combination yielded 2.0-4.4 log reductions for A. brasiliensis, whereas standalone ingredients, each ingredient yielded up to 1.1 log reductions on day 7. For E. coli, 6.6 log reductions on day 7 were observed for the combination, when only up to 3.1 log reductions were obtained as separate components.

Combination of Benzoin 0.055% and maltol 0.02%: The combination yielded 1.6-2.4 log reductions for A. brasiliensis, whereas standalone ingredients, each ingredient yielded up to 0.9 log reductions on day 7. For E. coli, 5.1 log reductions on day 14 were observed for the combination, when no log reductions were obtained as separate components.

### Example 2: Composition of Styrax Extract and Maltol in Cleanser Formulation

A Preservatives Effectiveness Test (PET) was carried out for various combinations of Styrax extract (Sumatra benzoin) and maltol (obtained from sugarcane fermentation) for evaluating the synergistic antimicrobial effect therebetween in a cleanser formulation. The antimicrobial efficiency was tested in accordance with current ISO 11930 requirements. The test consists of challenging a reference cleanser with a prescribed inoculum of suitable microorganisms. The tested microorganisms used for the PET are detailed in Table 5, while the various combinations of Styrax extract and maltol are detailed in Table 6.

TABLE 5
Tested microorganisms

| | | Culture | Culture Growth | PET Growth |
|---|---|---|---|---|
| Microorganism | ATCC | Media | Conditions | Conditions |
| Staphylococcus aureus (SA) | 6538 | TSA | 18-24 hr, | 3-5 days, |
| Escherichia coli | 8739 | | 32.5 ± 2.50 C. | 32.5 ± 2.50 C. |
| (EC) | | | | |
| Pseudomonas aeruginosa | 9027 | | | |
| (PA) | | | | |
| Candida albicans | 10231 | SDA | 48-72 hr, | 5-7 days, |
| (CA) | | | 22.5 ± 2.50 C. | 22.5 ± 2.50 C. |
| Aspergillus brasiliensis | 16404 | | 7 days, | |
| (AB) | | | 22.5 ± 2.50 C. | |

TABLE 6
wt % of Styrax extract and maltol in cleanser formulation

| Formula No. | Styrax ext. | Maltol |
|---|---|---|
| 1 | 5.5 | 2 |
| 2 | 5.5 | — |
| 3 | 5.5 | 0.2 |
| 4 | — | 2 |
| 5 | 0.55 | 2 |
| 6 | 2.75 | 1 |
| 7 | 2.75 | — |
| 8 | 2.75 | 0.2 |
| 9 | — | 1 |
| 10 | 0.55 | 1 |
| 11 | — | 0.2 |
| 12 | 0.55 | 0.2 |
| 13 | 0.55 | — |
| 14 | 0.55 | 0.04 |
| 15 | — | 0.04 |
| 16 | 0.11 | 0.2 |
| 17 | 0.11 | — |
| 18 | 0.11 | 0.04 |
| 19 | 0.55 | 0.02 |
| 20 | — | 0.02 |
| 21 | 0.055 | 0.2 |
| 22 | 0.055 | — |
| 23 | 0.055 | 0.02 |

| | |
|---|---|
| 24 | 1% Synthetic preservative (positive control) |
| 25 | Cleanser without additives (negative control) |

Each container was inoculated at time “0” and mixed with one of the microbial suspensions using a ratio-equivalent to 1% inoculum to product (30 μl inoculum into 3 g sample). The concentrations of test microorganisms added to the product were such that the concentration in the product immediately after inoculation is between 1×10⁶-1×10⁷ CFU/g for bacteria and 1×10⁵ 1×10⁶ CFU/g for molds. Samples from the challenged test product were drawn at specific time intervals and analyzed for microbial counts.

Samples were stored at 20-25° C. Each sample was sampled in intervals of 7, 14 and 28 days. At Day 7, 0.1 g of the sample were directly seeded on TSA plates for bacteria and on SDA plates for mold. For A. brasiliensis, a dilution of 1:10 was prepared (0.1 g of the inoculated lotion in 900 μL NaCl-peptone saline) and 0.1 μL of the 1:10 dilution was seeded on SDA plate. At Days 14 and 28, 0.1 g of the samples were directly seeded on TSA plates for bacteria and on SDA plates for molds.

The number of viable microorganisms/g in the mixture at each test interval was determined by the Spreading on agar Count Method. The TSA plates were incubated at 30-35° C. for 3-5 days unless a reliable count was obtained in a shorter time. The SDA plates were incubated at 20-25° C. for 5-7 days or less if reliable count was obtained prompter.

The counts obtained in all countable dilutions were used to calculate the mean value of CFU, per gram product/challenge microorganism/time interval. Using the calculated concentration of microorganisms in the initial inoculum per gram present as a baseline, the change in log 10 values of the concentration of the CFU/g was calculated for each time interval and the changes were expressed in terms of log reduction.

The summary of the results for the tested combinations, expressed in log reduction values, is provided in Table 7-1. Results (log reduction) of each of Styrax extract and Maltol are provided in Table 7-2 for comparison. In the Tables below, AB, CA, PA, EC and SA indicate A. brasilliensis, C. albicans, P. aeruginosa, E. coli and S. aureus, respectively.

TABLE 7-1
Summary of results for Styrax extract (benzoin) and maltol combinations,
log reduction from initial inoculation, in cleanser formulation

| Formula | | | 7 Days | | | | | 14 Days | | | | | 28 Days | | | | |
|---|---|---|---|---|---|---|---|---|---|---|---|---|---|---|---|---|---|
| # | BZ (wt %) | ML (wt %) | AB | CA | PA | EC | SA | AB | CA | PA | EC | SA | AB | CA | PA | EC | SA |
| 1 | 5.5 | 2 | 3.7 | 3.5 | 4.1 | 4.0 | 4.9 | 3.7 | 3.5 | 4.1 | 4.0 | 4.9 | 3.7 | 3.5 | 4.1 | 4.0 | 4.9 |
| 2 | 5.5 | — | 3.7 | 3.5 | 4.1 | 4.0 | 4.9 | 3.7 | 3.5 | 4.1 | 4.0 | 4.9 | 3.7 | 3.5 | 4.1 | 4.0 | 4.9 |
| 3 | 5.5 | 0.2 | 3.7 | 3.5 | 4.1 | 4.0 | 4.9 | 3.7 | 3.5 | 4.1 | 4.0 | 4.9 | 3.7 | 3.5 | 4.1 | 4.0 | 4.9 |
| 4 | — | 2 | 2.4 | 3.5 | 4.1 | 4.0 | 4.9 | 3.7 | 3.5 | 4.1 | 4.0 | 4.9 | 3.7 | 3.5 | 4.1 | 4.0 | 4.9 |
| 5 | 0.55 | 2 | 3.7 | 3.5 | 4.1 | 4.0 | 4.9 | 3.7 | 3.5 | 4.1 | 4.0 | 4.9 | 3.7 | 3.5 | 4.1 | 4.0 | 4.9 |
| 6 | 2.75 | 1 | 3.7 | 3.5 | 4.1 | 4.0 | 4.9 | 3.7 | 3.5 | 4.1 | 4.0 | 4.9 | 3.7 | 3.5 | 4.1 | 4.0 | 4.9 |
| 7 | 2.75 | — | 3.7 | 3.5 | 4.1 | 0.0 | 4.9 | 3.7 | 3.5 | 4.1 | 0.0 | 4.9 | 3.7 | 3.5 | 4.1 | 0.0 | 4.9 |
| 8 | 2.75 | 0.2 | 3.0 | 3.5 | 4.1 | 4.0 | 4.9 | 3.7 | 3.5 | 4.1 | 4.0 | 4.9 | 3.7 | 3.5 | 4.1 | 4.0 | 4.9 |
| 9 | — | 1 | 1.8 | 3.5 | 4.1 | 1.9 | 4.9 | 3.4 | 3.5 | 4.1 | 4.0 | 4.9 | 3.4 | 3.5 | 4.1 | 4.0 | 4.9 |
| 10 | 0.55 | 1 | 3.4 | 3.5 | 4.1 | 4.0 | 4.9 | 3.7 | 3.5 | 4.1 | 4.0 | 4.9 | 3.7 | 3.5 | 4.1 | 4.0 | 4.9 |
| 11 | — | 0.2 | 1.4 | 3.5 | 1.9 | 0.0 | 4.9 | 2.1 | 3.5 | 4.1 | 0.0 | 4.9 | 2.1 | 3.5 | 4.1 | 0.0 | 4.9 |
| 12 | 0.55 | 0.2 | 2.2 | 3.5 | 4.1 | 1.7 | 4.9 | 3.7 | 3.5 | 4.1 | 2.6 | 4.9 | 3.7 | 3.5 | 4.1 | 2.6 | 4.9 |
| 13 | 0.55 | — | 2.3 | 3.5 | 0.0 | 0.0 | 4.9 | 3.4 | 3.5 | 0.0 | 0.0 | 4.9 | 3.4 | 3.5 | 0.0 | 0.0 | 4.9 |
| 14 | 0.55 | 0.04 | 2.3 | 3.5 | 0.0 | 0.0 | 4.9 | 3.0 | 3.5 | 0.0 | 0.0 | 4.9 | 3.0 | 3.5 | 0.0 | 0.0 | 4.9 |
| 15 | — | 0.04 | 2.0 | 3.5 | 0.0 | 0.0 | 4.9 | 2.7 | 3.5 | 0.0 | 0.0 | 4.9 | 2.7 | 3.5 | 0.0 | 0.0 | 4.9 |
| 16 | 0.11 | 0.2 | 2.3 | 3.5 | 4.1 | 0.0 | 4.9 | 3.4 | 3.5 | 4.1 | 0.0 | 4.9 | 3.4 | 3.5 | 4.1 | 0.0 | 4.9 |
| 17 | 0.11 | — | 2.2 | 3.5 | 0.0 | 0.0 | 4.9 | 2.9 | 3.5 | 0.0 | 0.0 | 4.9 | 2.9 | 3.5 | 0.0 | 0.0 | 4.9 |
| 18 | 0.11 | 0.04 | 2.2 | 3.5 | 0.0 | 0.0 | 4.9 | 3.1 | 3.5 | 0.0 | 0.0 | 4.9 | 3.1 | 3.5 | 0.0 | 0.0 | 4.9 |
| 19 | 0.55 | 0.02 | 2.0 | 3.5 | 0.0 | 0.0 | 4.9 | 3.0 | 3.5 | 0.0 | 0.0 | 4.9 | 3.0 | 3.5 | 0.0 | 0.0 | 4.9 |
| 20 | — | 0.02 | 1.8 | 3.5 | 0.0 | 0.0 | 4.9 | 2.1 | 3.5 | 0.0 | 0.0 | 4.9 | 2.1 | 3.5 | 0.0 | 0.0 | 4.9 |
| 21 | 0.055 | 0.2 | 2.2 | 3.5 | 4.1 | 0.0 | 4.9 | 3.7 | 3.5 | 4.1 | 3.7 | 4.9 | 3.7 | 3.5 | 4.1 | 3.7 | 4.9 |
| 22 | 0.055 | — | 2.1 | 3.5 | 0.0 | 0.0 | 4.9 | 2.7 | 3.5 | 0.0 | 0.0 | 4.9 | 2.7 | 3.5 | 0.0 | 0.0 | 4.9 |
| 23 | 0.055 | 0.02 | 2.2 | 3.5 | 0.0 | 0.0 | 4.9 | 3.4 | 3.5 | 0.0 | 0.0 | 4.9 | 3.4 | 3.5 | 0.0 | 0.0 | 4.9 |

| | | | | | | | | | | | | | | | | |
|---|---|---|---|---|---|---|---|---|---|---|---|---|---|---|---|---|
| 24 | Synthetic | 1.6 | 3.5 | 4.1 | 3.4 | 4.9 | 4.9 | 3.5 | 4.1 | 4.0 | 4.9 | 4.9 | 3.5 | 4.1 | 4.0 | 4.9 |
| | Preservative 1% | | | | | | | | | | | | | | | |
| 25 | Lotion 100% | 1.6 | 3.5 | 0.0 | 0.0 | 4.9 | 1.8 | 3.5 | 0.0 | 0.0 | 0.0 | 1.8 | 3.5 | 0.0 | 0.0 | 0.0 |

TABLE 7-2
Summary of results for each of Styrax extract (benzoin) and maltol, log reduction
from initial inoculation, in cleanser formulation (amounts in wt %)

| | 7 Days | | | | | 14 Days | | | | | 28 Days | | | | |
|---|---|---|---|---|---|---|---|---|---|---|---|---|---|---|---|
| | AB | CA | PA | EC | SA | AB | CA | PA | EC | SA | AB | CA | PA | EC | SA |
| Benzoin | 3.7 | 3.5 | 4.1 | 4.0 | 4.9 | 3.7 | 3.5 | 4.1 | 4.0 | 4.9 | 3.7 | 3.5 | 4.1 | 4.0 | 4.9 |
| 5.5% | | | | | | | | | | | | | | | |
| Benzoin | 3.7 | 3.5 | 4.1 | 0.0 | 4.9 | 3.7 | 3.5 | 4.1 | 0.0 | 4.9 | 3.7 | 3.5 | 4.1 | 0.0 | 4.9 |
| 2.75% | | | | | | | | | | | | | | | |
| Benzoin | 2.3 | 3.5 | 0.0 | 0.0 | 4.9 | 3.4 | 3.5 | 0.0 | 0.0 | 4.9 | 2.3 | 3.5 | 0.0 | 0.0 | 4.9 |
| 0.55% | | | | | | | | | | | | | | | |
| Benzoin | 2.2 | 3.5 | 0.0 | 0.0 | 4.9 | 2.9 | 3.5 | 0.0 | 0.0 | 4.9 | 2.2 | 3.5 | 0.0 | 0.0 | 4.9 |
| 0.11% | | | | | | | | | | | | | | | |
| Benzoin | 2.1 | 3.5 | 0.0 | 0.0 | 4.9 | 2.7 | 3.5 | 0.0 | 0.0 | 4.9 | 2.1 | 3.5 | 0.0 | 0.0 | 4.9 |
| 0.055% | | | | | | | | | | | | | | | |
| Maltol | 2.4 | 3.5 | 4.1 | 4.0 | 4.9 | 3.7 | 3.5 | 4.1 | 4.0 | 4.9 | 3.7 | 3.5 | 4.1 | 4.0 | 4.9 |
| 2% | | | | | | | | | | | | | | | |
| Maltol | 1.8 | 3.5 | 4.1 | 1.9 | 4.9 | 3.4 | 3.5 | 4.1 | 4.0 | 4.9 | 3.4 | 3.5 | 4.1 | 4.0 | 4.9 |
| 1% | | | | | | | | | | | | | | | |
| Maltol | 1.4 | 3.5 | 1.9 | 0.0 | 4.9 | 2.1 | 3.5 | 4.1 | 0.0 | 4.9 | 2.1 | 3.5 | 4.1 | 0.0 | 4.9 |
| 0.2% | | | | | | | | | | | | | | | |
| Maltol | 2.0 | 3.5 | 0.0 | 0.0 | 4.9 | 2.7 | 3.5 | 0.0 | 0.0 | 4.9 | 2.7 | 3.5 | 0.0 | 0.0 | 4.9 |
| 0.04% | | | | | | | | | | | | | | | |
| Maltol | 1.8 | 3.5 | 0.0 | 0.0 | 4.9 | 2.1 | 3.5 | 0.0 | 0.0 | 4.9 | 2.1 | 3.5 | 0.0 | 0.0 | 4.9 |
| 0.02% | | | | | | | | | | | | | | | |

As can be seen from Tables 7-1 and 7-2, when examining the effect of Styrax extract alone, no activity against P. aeruginosa from 0.55%-0.055% and against E. coli from 2.75%-0.055%.

The effects obtained for Maltol alone was no activity against E. coli at 0.02-0.2% and against P. aeruginosa at 0.02-0.04%.

Combination of Benzoin 0.55% and Maltol 0.2%: The combination yielded 4.1 log reductions for P. aeruginosa at 7 days, whereas standalone ingredients, each ingredient yielded 0-1.9 log reductions.

Combination of Benzoin 0.11% and maltol 0.2%: The combination yielded 4.1 log reductions for P. aeruginosa whereas standalone ingredients, each ingredient yielded 0-1.9 log reductions at time 7.

Combination of Benzoin 2.75% and maltol 0.2%: The combination yielded 4.0 log reductions for E. coli whereas standalone ingredients, each ingredient yielded 0 log.

### Example 3: Composition of Maltol and Styrax Extract (Benzoin Extract), with and without Cinnamomum cassia Extract in Agar Dilution Test

An agar dilution test was carried out for various combinations of Styrax extract (Sumatra benzoin) in MCT oil and maltol (obtained from sugarcane fermentation), with and without Cinnamomum cassia extract (cassia oil) for evaluating the synergistic antimicrobial effect therebetween.

The agar dilution test was carried out as follows: TSA (tryptic soy agar) or SDA (Sabouraud dextrose agar) media were melted at 99±2° C. and kept warm until seeding (43±2° C.). Into a quota of the growth medium, the maltol and extract were added and stirred. 5 ml of the mixture was poured into each test plate and left to cool and solidify. The plates were then inoculated by defined amounts of test microorganisms (E. coli, S. aureus and P. aeruginosa used on TSA plates, C. albicans and A. brasilliensis used on the SDA plates). The plates were incubated at 25-35° C. for 24-48 hours and then assessed for microorganisms' growth. The agar dilution test provides test results which are independent of the formulation (e.g. cosmetic formulation) into which the composition is to be incorporated, hence can be used as a good basis for comparing activity of the compositions.

The test results are detailed in Table 8. (+) indicates growth, (+/−) indicates partial growth, while (−) indicates no growth. Positive control was obtained by utilizing a commercial preservative.

TABLE 8
Agar dilution test results for compositions of Styrax
extract (BZ) and maltol (amounts in wt %)

| | Test 1 | | | | | Teat 2 (duplicate) | | | | |
|---|---|---|---|---|---|---|---|---|---|---|
| | AB | CA | PA | EC | SA | AB | CA | PA | EC | SA |
| BZ 0.2% | + | + | +/− | +/− | +/− | + | + | +/− | +/− | +/− |
| BZ 0.5% | + | +/− | +/− | +/− | +/− | + | +/− | +/− | +/− | +/− |
| Cinnamomum cassia | − | − | + | +/− | +/− | − | − | + | +/− | +/− |
| oil 0.002% | | | | | | | | | | |
| Maltol 0.05% | + | + | +/− | + | +/− | + | + | +/− | + | +/− |
| Maltol 0.08% | +/− | − | − | +/− | + | +/− | − | − | +/− | + |
| Maltol 0.05% + | +/− | − | +/− | +/− | +/− | +/− | − | +/− | +/− | +/− |
| BZ 0.5% | | | | | | | | | | |
| Maltol 0.05% + | − | − | +/− | +/− | +/− | − | − | +/− | +/− | +/− |
| BZ 0.5% + | | | | | | | | | | |
| cassia 0.002% | | | | | | | | | | |
| Maltol 0.08% + | − | − | − | +/− | +/− | − | − | − | +/− | +/− |
| BZ 0.2% | | | | | | | | | | |
| Maltol 0.08% + | − | − | − | − | +/− | − | − | − | − | +/− |
| BZ 0.2% + | | | | | | | | | | |
| cassia 0.002% | | | | | | | | | | |
| Maltol 0.08% + | − | − | − | − | +/− | − | − | − | − | +/− |
| BZ 0.5% | | | | | | | | | | |
| Maltol 0.08% + | − | − | − | − | +/− | − | − | − | − | +/− |
| BZ 0.5% + | | | | | | | | | | |
| cassia 0.002% | | | | | | | | | | |
| Maltol 0.2% + | − | − | − | − | − | − | − | − | − | − |
| BZ 0.5% + | | | | | | | | | | |
| cassia 0.002% | | | | | | | | | | |
| Positive | − | − | − | − | − | − | − | − | − | − |
| control | | | | | | | | | | |
| Negative | + | + | + | + | + | + | + | + | + | + |
| control | | | | | | | | | | |

As can be seen, a composition of 0.2 wt % maltol, 0.5% benzoin and 0.002% cassia extract showed antimicrobial activity against all tested microorganisms. For E. coli, a clear synergistic effect can be observed for a composition of 0.08 wt % maltol, 0.2 wt % benzoin and 0.002 wt % cassia extract, while for both C. Albicans and A. brasiliensis a clear synergistic effect is demonstrated for a composition of 0.05 wt % maltol and 0.5 wt % benzoin.

### Example 4: Composition of Maltol and Cinnamomum cassia Extract

A Preservatives Effectiveness Test (PET) was carried out for various combinations of Cinnamomum cassia extract (cassia oil) and maltol (obtained from sugarcane fermentation) for evaluating the synergistic antimicrobial effect therebetween in lotion formulation, according to the protocol of Example 1. The results are provided in Table 9.

TABLE 9
Summary of results for maltol (ML) and Cinnamomum cassia extract (CN), log
reduction from initial inoculation, in lotion formulation (amounts in wt %)

| Formula | | | 7 Days | | | | 14 Days | | | | 28 Days | | | |
|---|---|---|---|---|---|---|---|---|---|---|---|---|---|---|
| # | CN (wt %) | ML (wt %) | AB | CA | SA | PA | AB | CA | SA | PA | AB | CA | SA | PA |
| 1 | 0.02 | — | 5.5 | 5.1 | 0 | 4.2 | 5.5 | 5.1 | 6.3 | 0 | 5.5 | 5.1 | 6.3 | 0 |
| 2 | 0.01 | — | 0 | 0 | 0 | 0 | 0 | 0 | 4.1 | 0 | 0 | 0 | 6.3 | 0 |
| 3 | 0.002 | — | 1.2 | 0 | 0 | 0 | 0 | 0 | 0 | 0 | 0 | 0 | 6.3 | 0 |
| 4 | 0.0004 | — | 0 | 0 | 0 | 0 | 0 | 0 | 0 | 0 | 0 | 0 | 0 | 0 |
| 5 | 0.0002 | — | 0 | 0 | 0 | 0 | 0 | 0 | 2.8 | 0 | 0 | 0 | 6.3 | 0 |
| 6 | — | 1.2 | 5.5 | 5.1 | 6.3 | 6.7 | 5.5 | 5.1 | 6.3 | 6.7 | 5.5 | 5.1 | 6.3 | 3.3 |
| 7 | — | 0.6 | 5.5 | 5.1 | 2.4 | 0 | 5.5 | 5.1 | 6.3 | 6.7 | 3.5 | 5.1 | 6.3 | 3.2 |
| 8 | — | 0.12 | 1.9 | 5.1 | 0 | 0 | 1.8 | 5.1 | 3.6 | 0 | 0 | 5.1 | 6.2 | 0 |
| 9 | — | 0.024 | 0 | 0 | 0 | 0 | 0 | 0 | 2.1 | 0 | 0 | 0 | 6.3 | 0 |
| 10 | — | 0.012 | 0 | 0 | 0 | 0 | 0 | 0 | 0 | 0 | 0 | 0 | 0 | 0 |
| 11 | 0.02 | 1.2 | 5.5 | 5.1 | 6.3 | 6.7 | 5.5 | 5.1 | 4.3 | 6.7 | 5.5 | 5.1 | 6.3 | 6.7 |
| 12 | 0.02 | 0.12 | 5.5 | 5.1 | 2.5 | 0 | 5.5 | 5.1 | 6.3 | 0 | 5.5 | 5.1 | 6.3 | 0 |
| 13 | 0.002 | 1.2 | 5.5 | 5.1 | 6.3 | 6.7 | 5.5 | 5.1 | 6.3 | 6.7 | 5.5 | 5.1 | 6.3 | 3.6 |
| 14 | 0.01 | 0.6 | 5.5 | 5.1 | 2.3 | 0 | 5.5 | 5.1 | 6.3 | 6.7 | 5.5 | 5.1 | 6.3 | 3.3 |
| 15 | 0.01 | 0.12 | 5.5 | 2.1 | 1.8 | 0 | 5.5 | 5.1 | 6.3 | 0 | 2.5 | 5.1 | 6.3 | 0 |
| 16 | 0.002 | 0.6 | 5.5 | 5.1 | 2.9 | 0 | 5.5 | 5.1 | 6.3 | 6.7 | 5.5 | 5.1 | 6.3 | 3.2 |
| 17 | 0.002 | 0.12 | 1.5 | 5.1 | 0 | 0 | 2.2 | 5.1 | 4.1 | 0 | 0 | 5.1 | 6.3 | 0 |
| 18 | 0.002 | 0.024 | 1.9 | 0 | 0 | 0 | 0 | 0 | 3.9 | 0 | 0 | 0 | 6.2 | 0 |
| 19 | 0.0004 | 0.12 | 2.1 | 5.1 | 0 | 0 | 2.1 | 5.1 | 3.4 | 0 | 0 | 5.1 | 6.2 | 0 |
| 20 | 0.0004 | 0.024 | 0 | 0 | 0 | 0 | 0 | 0 | 3.1 | 0 | 0 | 0 | 6.2 | 0 |
| 21 | 0.002 | 0.012 | 1.5 | 0 | 0 | 0 | 0 | 0 | 0 | 0 | 0 | 0 | 6.2 | 0 |
| 22 | 0.0002 | 0.12 | 1.7 | 5.1 | 0 | 0 | 0 | 5.1 | 2.9 | 0 | 0 | 5.1 | 4.6 | 0 |
| 23 | 0.0002 | 0.012 | 0 | 0 | 0 | 0 | 0 | 0 | 2.6 | 0 | 0 | 0 | 6.2 | 0 |

| | | | | | | | | | | | | | |
|---|---|---|---|---|---|---|---|---|---|---|---|---|---|
| 24 | Synthetic | 5.5 | 5.1 | 6.3 | 6.7 | 5.5 | 5.1 | 6.3 | 6.7 | 5.5 | 5.1 | 6.3 | 2.8 |
| | Preservative 1% | | | | | | | | | | | | |
| 25 | Lotion 100% | 0 | 0 | | | 0 | 0 | | | 0 | 0 | | |

As can be seen, synergistic antimicrobial activity against C. Albicans and A. brasiliensis is obtained starting from compositions containing 0.12 wt % maltol and 0.0004 wt % Cinnamomum cassia extract.

### Example 5: Compositions of Maltol and Various Extracts

An agar dilution tests were carried out for various combinations of maltol (obtained from sugarcane fermentation) and various extracts for evaluating the synergistic antimicrobial effect therebetween. The test protocol was the same as in Example 3.

The tested extracts were Malpighia emarginata (acerola) fruit extract, Thymus vulgaris (thyme) leaves extract, Origanum vulgare leaves (oregano) extract, Syzygium aromaticum (clove) bud oil, Nigella sativa seeds extract, Pimenta dioica fruit (allspice) extract, Terminalia ballerica fruit extract, Magnolia officinalis bark extract, Cymbopogon citratus (lemongrass) leaves extract, and Sapindus mukorossi saponin extract.

The test results are detailed in Table 10. (+) indicates growth, (+/−) indicates partial growth, while (−) indicates no growth. Positive control was obtained by utilizing a commercial preservative.

TABLE 10
Agar dilution test results for compositions of
maltol and various extracts (amounts in wt %)

Test results

| | AB | CA | PA | EC | SA |
|---|---|---|---|---|---|
| Maltol 0.1% | +/− | − | + | + | − |
| Acerola 0.2% | + | + | + | + | + |
| Maltol 0.1% + acerola 0.2% | +/− | − | − | +/− | − |
| Thyme 0.2% | +/− | − | + | + | − |
| Maltol 0.1% + thyme 0.2% | − | − | − | − | − |
| Oregano 0.2% | − | − | + | − | − |
| Maltol 0.1% + oregano 0.2% | − | − | − | − | − |
| Clove 0.2% | − | − | + | + | − |
| Maltol 0.1% + clove 0.2% | − | − | − | − | − |
| Nigella 0.2% | + | + | + | + | − |
| Maltol 0.1% + nigella 0.2% | − | − | − | − | − |
| Allspice 0.2% | − | − | + | + | − |
| Maltol 0.1% + allspice 0.2% | − | − | − | − | − |
| Terminalia 0.2% | + | + | + | + | + |
| Maltol 0.1% + terminalia | − | − | − | +/− | + |
| 0.2% | | | | | |
| Magnolia 0.2% | + | + | + | + | + |
| Maltol 0.1% + magnolia | +/− | − | − | +/− | − |
| 0.2% | | | | | |
| Lemongrass 0.2% | + | + | + | + | + |
| Maltol 0.1% + lemongrass | − | − | − | +/− | − |
| 0.2% | | | | | |
| Sapindus 0.2% | + | + | + | + | − |
| Maltol 0.1% + sapindus 0.2% | − | − | − | +/− | − |
| Positive control | − | − | − | − | − |
| Negative control | + | + | + | + | + |

As can be seen in Table 10, Maltol in 0.1 wt % concentration has antimicrobial activity against C. albicans and S. aureus and partial activity against A. brasiliensis.

When maltol is combined with different plant extracts, the following was observed:
Maltol and Acerola—synergism was observed in antimicrobial activity against P. aeuroginosa and partially for E. coli;
Maltol and Thyme—synergism was observed in antimicrobial activity against P. aeuroginosa, E. coli and A. Brasileinsis;
Maltol and Oregano—synergism was observed in antimicrobial activity against P. aeuroginosa;
Maltol and Clove—synergism was observed in antimicrobial activity against P.aeuroginosa, E. coli;
Maltol and Nigella—synergism was observed in antimicrobial activity against P. aeuroginosa, E. coli and A. Brasileinsis;
Maltol and Allspice—synergism was observed in antimicrobial activity against P. aeuroginosa, E. coli;
Maltol and Terminalia—synergism was observed in antimicrobial activity against P. aeuroginosa, A. Brasileinsis and partially E. coli.;
Maltol and Magnolia—synergism was observed in antimicrobial activity against P.aeuroginosa and partially E. coli;
Maltol and lemongrass-synergism was observed in antimicrobial activity against P.aeuroginosa, A. Brasileinsis and partially E. coli;
Maltol and Sapindus Mukorossi-synergism was observed in antimicrobial activity against P.aeuroginosa, A. Brasileinsis and partially E. coli.

### Example 6: Delayed antimicrobial activity

A Preservatives Effectiveness Test (PET) was carried out for a composition of 0.2 wt % maltol, 0.5 wt % Styrax extract (Sumatra benzoin) and 0.002% cassia oil was carried out according to the protocol of Example 1, however inoculated with 5×10⁶ CFU/ml of S. aureus (SA), 5×10⁶ CFU/ml of S. epidermis (SE) and a 6.3×10⁶ CFU/ml mixture of both. These microorganisms are indicative of microorganisms residing naturally on the skin and are part of the desired microbiota of the skin. The log reduction was recorded for various test points, as shown in Table 11.

TABLE 11
Summary of results for Styrax extract (benzoin) and maltol combinations,
log reduction from initial inoculation, in cleanser formulation

| | 7 hours | | | 24 hours | | | 48 hours | | | 7 days | | |
|---|---|---|---|---|---|---|---|---|---|---|---|---|
| Tested | | | 1:1 | | | 1:1 | | | 1:1 | | | 1:1 |
| composition | SA | SE | mix | SA | SE | mix | SA | SE | mix | SA | SE | mix |
| 0.2% maltol + | 0 | 0 | 0 | 0 | 0 | 0 | 0 | 0 | 0 | 6.69 | 6.70 | 6.80 |
| 0.5% BZ + | | | | | | | | | | | | |
| 0.002% cassia | | | | | | | | | | | | |
| Cream with | 1.54 | 6.70 | 1.62 | 4.51 | 6.70 | 6.80 | 6.69 | 6.70 | 6.80 | 6.69 | 6.70 | 6.80 |
| synthetic | | | | | | | | | | | | |
| preservative | | | | | | | | | | | | |
| Unpreserved | 0 | 0 | 0 | 0 | 0 | 0 | 0 | 0 | 4.25 | 2.15 | 6.7 | 2.7 |
| cream | | | | | | | | | | | | |

A can be seen, no reduction in microbial population was observed for the first 48 hours in a cream that contained the composition according to this disclosure, indicating that only after 48 hours the natural microbiota of the skin is expected to be affected. However, for the synthetically preserved cream, elimination of microbiota is clearly observable starting from several hours after exposure. Thus, the maltol/BZ/cassia composition, unlike the synthetic preservative, is not expected to hinder the natural microbiota for the time period that the formulation normally resides on the skin (several hours), however, as show in Examples 1-3 above, provides antimicrobial activity against the undesired microorganism in the cream.

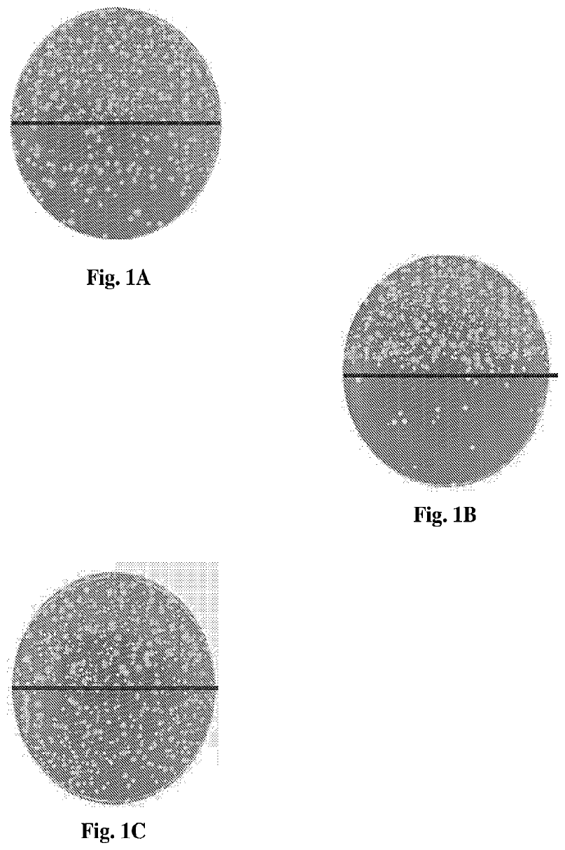

This selective effect is also demonstrated in FIGS. 1A-1C, which are pictures of an agar plate inoculated with a mixture of S. aureus, S. capitis, S. epidermis and S. hominis, taken after 7 hours of incubation. The mixture of microorganisms represents the natural skin microbiota. FIG. 1A shows a sample of cream without any preservatives. FIG. 1B shows a sample of the cream in which the bottom half of the sample contained a commercial synthetic preservative (ethylhexyl glycerin 1%) and the upper half did not contain a preservative-showing almost complete elimination of the microbiota in the preserved region. In comparison, FIG. 1C shows a sample of the cream with the bottom half of the sample containing the maltol/BZ/cassia composition, and without preservative in the upper half. Contrary to the commercial preservative, it can be seen that in the composition of this disclosure, no harm to the natural microbiota is observed, indicating the delayed antimicrobial activity of compositions of the disclosure.

1. An antimicrobial composition comprising at least one 4-pyrone derivative and at least one plant extract from at least one plant species.

2. The composition of claim 1, wherein said 4-pyrone derivative is obtained from a plant source different from said at least one plant species.

3. The composition of claim 1, wherein the at least one 4-pyrone derivative is a compound having a structure of formula (I): 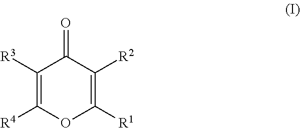
wherein:
R¹ is selected from H, C₁₋₆alkyl, C₁₋₆hydroxyalkyl, C₁₋₆acyl, and C₂₋₆alkenyl;
R² is selected from H, OH, —O—C(O)—R⁵;
R³ is H or OH;
R⁴ is H or OH;
R⁵ is selected from H, C₁₋₆alkyl, C₁₋₆hydroxyalkyl, and a phenyl (optionally substituted by one, two or three substituents independently selected from H, OH, halo, C₁₋₆alkyl, C₂₋₆alkenyl, and —O—C(O)—(C₁₋₆alkyl));
or a salt or a hydrate thereof,
provided that at any instance at least one of R¹, R², R³ and R⁴ is not H.

4. The composition of claim 3, wherein
R¹ is selected from H, C₁₋₆alkyl, C₁₋₆hydroxyalkyl, and C₁₋₆acyl;
R² is H or OH;
R³ is H or OH;
R⁴ is H or OH;
provided that at any instance at least one of R¹, R², R³ and R⁴ is not H.

5. The composition of claim 1, wherein the 4-pyrone derivative is selected from the group consisting of:
3-Hydroxy-2-methyl-4H-pyran-4-one (maltol),
5-Hydroxy-2-methyl-4H-pyran-4-one (allomaltol),
5-Hydroxy-2-(hydroxymethyl)-4H-pyran-4-one (kojic acid),
2-Ethyl-3-hydroxy-4H-pyran-4-one,
3-hydroxy-2-propylpyran-4-one,
3-hydroxy-2-propan-2-ylpyran-4-one,
2-acetyl-3-hydroxypyran-4-one,
2-ethyl-5-hydroxypyran-4-one,
3-hydroxy-2-(2-propen-1-yl)-4H-Pyran-4-one,
3-hydroxypyran-4-one,
2-Hex-2-enyl-3-hydroxypyran-4-one,
3,5-Dihydroxy-2-methyl-4H-pyran-4-one (5-Hydroxymaltol),
2-Methyl-4-oxo-4H-pyran-3-yl isobutyrate (Maltol isobutyrate),
2-Methyl-3-(1-oxopropoxy)-4H-pyran-4-one (Maltol propionate),
2-Methyl-4-oxo-4H-pyran-3-yl butyrate (Maltol butyrate),
(2-methyl-4-oxopyran-3-yl) acetate,
(2-ethyl-4-oxopyran-3-yl) 2-methylpropanoate,
2-Methyl-4-oxo-4H-pyran-3-yl 2-hydroxybenzoate (Salicylic acid maltol ester),
2-Methyl-4-oxo-4H-pyran-3-yl lactate (Maltol lactate),
2-Methyl-4-oxo-4H-pyran-3-yl 2-(acetyloxy) benzoate (Aspalatone),
(Z)-(2-Methyl-4-oxopyran-3-yl)-2-methylpent-2-enoate, and
(E)-(2-Methyl-4-oxopyran-3-yl)-2-methylpent-2-enoate;
and any combination thereof.

6. The composition of claim 5, wherein the 4-pyrone derivative is selected from the group consisting of maltol, 5-hydroxy-2-(hydroxymethyl)-4H-pyran-4-one (kojic acid), and 2-ethyl-3-hydroxy-4H-pyran-4-one.

7. The composition of claim 6, wherein the 4-pyrone derivative is maltol.

8.-9. (canceled)

10. The composition of claim 1, wherein the composition comprises at least one 4-pyrone derivative in a content of between about 0.001 and about 5 wt %.

11. (canceled)

12. The composition of claim 1, wherein said at least one plant species is selected from saponin-containing plant material.

13. The composition of claim 12, wherein said saponin-containing plant material is selected from at least one of Camellia oleifera, Camellia sinensis, Quillaja saponaria, Sapindus mukorossi, Sapindus saponaria, and Saponaria officinalis.

14. (canceled)

15. The composition of claim 1, wherein said at least one plant extract is selected from the group consisting of Styrax paralleloneurus resin and/or bark extract, Styrax tonkinensis resin and/or bark extract, Styrax tolu resin and/or bark extract, Myroxylon balsamum resin and/or bark extract, Myroxylon peruiferum resin and/or bark extract, Myrocarpus fastigiatus resin and/or bark extract, Nigella sativa seeds extract, Cuminum cyminum seeds extract, Zingiber officinale root or bulb extract, Cinnamomum cassia bark or twig extract, Paeonia lactiflora root extract, Terminalia ballerica or Terminalia chebula leaves, fruit, or pericarp extract, Commiphora myrrha resin extract, Boswellia serrata resin extract, Dipterocarpus turbinatus resin extract, Copaiba langsdorffii resin or bark extract, Malpighia glabra fruit extract, Thymus vulgaris leaves extract, Origanum vulgare leaves extract, Cymbopogon citratus leaves extract, Anethum graveolens seeds extract, Syzygium aromaticum flowers or buds extract, Magnolia officinalis bark extract, Malpighia emarginate fruit extract, Malpighia glabra fruit extract, Pimenta dioica fruit extract, Salvia officinalis leaves extract, and mixtures thereof.

16. (canceled)

17. The composition of claim 1, wherein the weight ratio between the at least one 4-pyrone derivative and the at least one plant extract is between about 1:1 to about 100:1.

18. The composition of claim 1, wherein the weight ratio between the at least one 4-pyrone derivative and the at least one plant extract is between about 1:10 to about 10:1.

19. An add-on antimicrobial composition consisting of a combination of:
at least one 4-pyrone derivative; and
at least one plant extract from at least one plant species selected from the group consisting of the genera Styrax, Myroxylon, Myrocarpus, Nigella, Cuminum, Zingiber, Cinnamomum, Paeonia, Terminalia, Commiphora, Boswellia, Dipterocarpus, Copaiba, Thymus, Origanum, Cymbopogon, Anethum, Syzygium, Magnolia, Malpighia, Pimenta, Salvia, and from a saponin-containing plant material, and any mixtures or combinations thereof.

20. (canceled)

21. A The composition of claim 1 having an antimicrobial activity, selected from the group consisting of:
(a) a composition comprising Styrax extract and maltol;
(b) a composition comprising Styrax extract, maltol, and Nigella sativa extract;
(c) a composition comprising Styrax extract, maltol, and Zingiber officinale extract;
(d) a composition comprising Styrax extract, maltol, and Cinnamomum cassia extract;
(e) a composition comprising Styrax extract, maltol, Cinnamomum cassia extract, and citric acid;
(f) a composition comprising Styrax extract, maltol, Paeonia lactiflora extract, and Terminalia ballerica extract; and
(g) a composition comprising Styrax extract, maltol, Paeonia lactiflora extract, Terminalia ballerica extract and citric acid;
(h) a composition comprising maltol and Paeonia lactiflora extract;
(i) a composition comprising maltol and Terminalia ballerica extract;
(j) a composition comprising maltol and Magnolia officinalis extract;
(k) a composition comprising maltol and Cymbopogon citratus (lemongrass) extract;
(l) a composition comprising maltol and Salvia officinalis leaves extract;
(m) a composition comprising maltol and Nigella sativa extract;
(n) a composition comprising maltol and Pimenta dioica (allspice) extract;
(o) a composition comprising maltol and Cinnamomum cassia extract;
(p) a composition comprising maltol and Malpighia emarginata (acerola) extract;
(q) a composition comprising maltol and Origanum vulgare extract;
(r) a composition comprises maltol and Sapindus mukorossi extract;
(s) a composition comprising maltol and Camelia oleifera extract;
(t) a composition comprising maltol and Syzygium aromaticum (clove) extract; and
(u) a composition comprising maltol and Thymus vulgaris extract.

22. The composition of claim 1, having a delayed antimicrobial activity.

23. The composition of claim 22, wherein the composition shows antimicrobial activity observable from at least 5 hours of continuous exposure of microorganisms to the composition.

24. The composition of claim 1, formulated as a preservative formulation, an antimicrobial formulation, a pharmaceutical composition, a disinfectant formulation, an oral care formulation, or a cosmetic formulation.

25. The composition of claim 24, being a cosmetic product selected from the group consisting of a cosmetic product, a skin care product, a hair care product, and an oral care product.

26. A The preservative formulation comprising the composition as defined in claim 1.

27. (canceled)
